(19) United States
(12) Patent Application Publication
(10) Pub. No.: US 2021/0102260 A1
(43) Pub. Date: Apr. 8, 2021

(54) PATIENT CLASSIFICATION AND PROGNOSITIC METHOD

(71) Applicants: The Institute of Cancer Research: Royal Cancer Hospital, London, Greater London (GB); The Royal Marsden NHS Foundation Trust, London, Greater London (GB); ARC-NET Centre for Applied Research on Cancer Universita Degli Studi Di Verona, Verona (IT)

(72) Inventors: Anguraj Sadanandam, London, Greater London (GB); Gift Nyamundanda, London, Greater London (GB); Kate Young, London, Greater London (GB); Aldo Scarpa, Verona (IT); Chanthirika Ragulan, London, Greater London (GB)

(21) Appl. No.: 16/970,178

(22) PCT Filed: Feb. 15, 2019
(86) PCT No.: PCT/EP2019/053845

§ 371 Date: Aug. 14, 2020

(30) Foreign Application Priority Data
Feb. 16, 2018 (EP) 18425009.0

(51) Int. Cl. C12Q 1/6886; G16B 25/10

(52) U.S. Cl. CPC C12Q 1/6886; C12Q 2600/158; C12Q 2600/118; G16B 25/10

(57) ABSTRACT

The present invention relates to methods for predicting prognosis and overall survival among tumour/cancer patients, and methods for classifying and stratifying these patients, particularly patients having pancreatic neuroendocrine tumors (PanNETs). The invention also relates to therapeutic methods for treating classified patients. Measuring gene expression levels of at least some of a selected group 198 genes is shown to be useful in the stratification of patients into groups with prognostic significance, and making a prediction of prognosis.

### FIELD OF THE INVENTION

The present invention relates to materials and methods for predicting prognosis and overall survival among tumor/cancer patients, and to methods for stratifying these patients, particularly patients having pancreatic neuroendocrine tumors (PanNETs).

### BACKGROUND TO THE INVENTION

Neuroendocrine tumors (NETs) are rare and heterogeneous tumors with widely varying morphologies and behaviours. As such, progress in improving their treatment has been slow. However, there have been recent advances in their characterisation, our understanding of their underlying biology and in the treatment options available¹.

NETs arise in multiple organs but over 65% occur in the GI tract, known as GEP-NETs², of which pancreatic neuroendocrine tumors (PanNETs) are a sub-group. Whilst GEP-NETs remain a rare cancer their incidence has significantly increased to 5.25/100,000/year, according to Surveillance, Epidemiology and End Results (SEER) program data³.

Overall survival (OS) for PanNETs is in the order of 99 months⁴. 5-year survival for PanNETs ranges from 60-100% for localised disease to 25% for metastatic⁵. Whilst relatively good in oncological terms these prognoses remain life-limiting for the majority and significantly worse for many patients.

In 2010 the World Health Organisation (WHO) classified Neuroendocrine Neoplasms (NENs) according to various histopathological features and the tumor's proliferative index, assessed by Ki67%. The main division was between well and poorly differentiated tumors; the former grouped as Grade 1/2 NETs and the latter labelled Grade 3 Neuroendocrine Carcinomas (NECs). The grades have prognostic significance with Grade 1 tumors (Ki67<3%) having the best prognosis and Grade 3 tumors (Ki67>20%) the worst^{6,4}.

The treatment paradigm for PanNETs is largely based upon these grades, alongside tumor site and functionality, as there are no other validated prognostic and/or predictive biomarkers routinely used in clinical practice^{7,8,9,10}.

Surgery is the only curative treatment, but as patients frequently present with advanced disease this is often impossible. Patients with Grade 1/2 disease are treated with a less aggressive approach, often initially with watchful waiting/somatostatin analogues before more intensive treatment when initial treatment fails. Patients with Grade 3 disease tend to be treated more aggressively with immediate platinum-based chemotherapy doublets.

However, there is significant heterogeneity of disease behaviour within grades, as suggested by recent literature and our clinical experience^{11,12,13,14}. This heterogeneity has in part been recognised by the WHO, who published an update to their classification in 2017, adding a 3^{rd} well differentiated NET subgroup, NET Grade 3¹⁵.

TABLE 1
A Comparison of the WHO Classification of GEP-NETs from
2010 and 2017

| WHO 2010 | | | WHO 2017 | | |
|---|---|---|---|---|---|
| Differentiation | Grade | Ki67 | Differentiation | Grade | Ki67 |
| Well | NET | <3% | Well | NET | <3% |
| Differentiated | Grade 1 | | Differentiated | Grade 1 | |
| NET | NET | 3-20% | NEN | NET | 3-20% |
| | Grade 2 | | | Grade 2 | |
| | | | | NET | >20% |
| | | | | Grade 3 | |
| Poorly | NEC | >20% | Poorly | NEC | >20% |
| Differentiated | Grade 3 | | Differentiated | Grade 3 | |
| NEC | (small | | NEN | (small | |
| | cell or | | | cell or | |
| | large | | | large | |
| | cell) | | | cell) | |

In the clinic the heterogeneity of behaviour within grades may manifest as a patient having a lower grade tumor (1/2) which behaves more like a Grade 3 tumor and perhaps should be treated aggressively upfront and vice versa. However, there is no strong evidence base to determine which patients require treatment intensification or indeed de-escalation, sparing them unnecessary treatment and attendant side effects.

There is an unmet clinical need for prognostic and predictive biomarkers and clinically-relevant assays to complement or replace grade and improve PanNET patient stratification, classification and prognosis.

### BRIEF DESCRIPTION OF THE INVENTION

The present invention is based on an investigation to identify biomarkers used to stratify PanNETs into molecular subtypes with distinct prognosis.

The inventors have identified biomarkers associated with overall survival (OS). The identified biomarkers are independent of the grades previously used by WHO to classify patients and inform treatment choices.

In particular, gene expression levels of a selected group of 198 genes were shown to be useful in the stratification of patients into groups with prognostic significance.

Additionally, mutations (targeted mutational profiles of MEN1, DAXX/ATRX, TSC2, PTEN and ATM) may be used to stratify/classify patients into groups which are associated with different prognoses.

Thus, the present invention provides a novel low-cost multiplex biomarker assay to stratify PanNETs into molecular subtypes with distinct prognoses.

Various groups have sought to describe the molecular nature of PanNETs, with whole-genome analysis recently published¹⁶. Recurrent gene alterations have been described in four main pathways in sporadic PanNETs, telomere maintenance (DAXX/ATRX), chromatin remodelling (SETD2, ARID1A, MLL3), mTOR pathway activation (PTEN, TSC1/2, DEPDC5) and DNA damage repair (CHEK2, BRCA2, MUTYH, ATM) with MEN1 inactivation influencing all four pathways^{17,16}.

Attempts have been made to associate these and other mutations with prognosis or treatment response but the majority of studies have been small and retrospective in nature and strong conclusions cannot yet be drawn¹⁸. For example, DAXX/ATRX mutations and alternative lengthening of telomeres (ALT) have been associated with a poor prognosis across a number of studies^{16,19,20} but an improved prognosis in others^{17,21}.

Three molecular subtypes in sporadic PanNETs have been previously identified by the lab, based on an integrated analysis of gene expression (221 genes), microRNA (30 miRs) and mutations (targeted mutational profiles of MEN1, DAXX/ATRX, TSC2, PTEN and ATM), collectively named the PanNETassigner signature²². The existence of three subtypes was supported by Scarpa et al. who reported three similar subtypes using RNA-sequencing¹⁶.

The three PanNETassigner subtypes, Metastasis-like-primary (MLP), Insulinoma-like and Intermediate identified each have specific features. Their prognostic significance has not previously been assessed.

TABLE 2
PanNETassigner Molecular Subtypes
PanNETassigner Subtypes

| MLP | Insulinoma-like | Intermediate |
|---|---|---|
| 38% of patients | 25% of patients | 37% of patients |
| usually non | usually functional | usually non |
| functional | | functional |
| high metastatic | low metastatic | moderate metastatic |
| potential | potential | potential |
| grade 1/2/3 | grade 1/2 | grade 1/2 |
| DAXX, ATRX, TSC2, | TSC2, PTEN, ATM | MEN1, DAXX/ATRX |
| PTEN, ATM mutations | mutations | mutations |

Grade 1/2 PanNETs are heterogeneous, associated with all three molecular subtypes, whereas Grade 3 tumors are predominantly associated with the MLP subtype.

The present invention provides methods of classifying/stratifying PanNETs into molecular subtypes which the inventors have identified as having distinct prognoses. The present invention provides methods of predicting prognosis based on the classification/stratification of PanNETs into molecular subtypes which the inventors have identified as having distinct prognoses. The identified biomarkers can be used independently to the grade system previously used by WHO to classify patients and inform treatment choices. The identified biomarkers provide additional prognostic information as compared to the grade system. The identified biomarkers can therefore be alongside and in addition to the grade system.

Prognosis can be predicted using gene expression levels of some or all of a group 198 genes shown in table 5; and the mutation status of MEN1, DAXX/ATRX, TSC1, TSC2, PTEN and ATM.

Accordingly, the invention relates to the use of these biomarkers (gene expression and optionally mutations) for stratifying/classifying patients with PanNETs and predicting the prognosis of a patient with a PanNET.

The invention also relates to methods for identifying patients for treatment, and to methods of treatment of PanNETs.

In a first aspect, the invention relates to a method for predicting the prognosis of a human pancreatic neuroendocrine tumor (PanNET) patient, the method comprising:
a) measuring the gene expression of at least 30 genes selected from: CEACAM1, INS, PFKFB2, ELSPBP1, MIA2, ENTPD3, GRM5, STEAP3, APOH, SERPINA1, A1CF, PRLR, F10, TMEM176B, MASP2, RBP4, CYP4F3, CHST8, KLK4, USP29, CELA1, TM4SF4, TMPRSS4, SCD5, TM4SF5, SERPIND1, P2RX1, GLP1R, LRAT, CASR, DAPL1, ERBB3, C19orf77, F7, PLIN3, NEFM, MNX1, ROBO3, CPA1, CTRL, TGFBR3, PNLIPRP2, TSHZ3, ADAMTS2, GLRA2, HGD, GP2, CTRC, RAB17, ANGPTL3, LOXL4, PNLIP, PEMT, CPA2, PNLIPRP1, ALDH1A1, SLC12A7, IL20RA, CLPS, GLS, C20orf46, GCGR, IL18R1, PDIA2, NAAA, BTC, TAPBPL, ELMO1, KLK8, CDS1, TFF1, TBC1D24, KIT, MOBKL1A, PLA1A, SUSD5, CRYBA2, PMM1, EFNA1, SLC16A3, FKBP11, IL22RA1, ADM, EGLN3, LGALS4, TLE2, CLDN10, NUPR1, SERPINI2, PTPLA, PVRL4, EGFR, MAFB, PFKFB3, HSD11B2, FGB, NDC80, SMOC2, ACVR1B, TGIF1, ARRDC4, MMP1, TACSTD2, TOP2A, SH3BP4, PDGFC, THBS2, CNPY2, HAO1, ADAM28, C7orf68, GATM, CXCR4, PAFAH1B3, NEK6, AKR1C4, F12, PMEPA1, RAB7L1, SMO, CLDN1, CHST1, WNT4, TMPRSS15, SPAG4, MX2, SLC7A2, GUCA1C, SLC7A8, PRSS22, RARRES2, PRSS8, SLC30A2, TMEM90B, VIPR2, CXCR7, SMARCA1, FAM19A5, CLDN11, SERPINA3, GAL3ST4, AFG3L1, COL8A1, SSX2IP, IMPA2, VEGFC, TMEM181, LGALS2, PLXDC1, TLR3, PSMB9, CHI3L2, PLCE1, ABI3BP, NUDT5, FOXO4, SLC2A1, COL1A2, REG1B, NETO2, ENC1, DLL1, TM4SF1, CKS2, FGD1, PPEF1, LEF1, MLN, TNFAIP6, ACAD9, TYMS, ZNF521, ACADSB, TSC2, HR, DEFB1, GRSF1, ACE, SRGAP3, SMEK1, TWIST1, FMNL1, ADAMTS7, COL5A2, IFI44, CAPN13, AQP8, IP6K2, COPE, MXRA5, RBPJL, MBP, MAP3K14, CLCA1, IDS, TECR, CAPNS1 and POSTN, in a sample obtained from the PanNET of the patient to obtain a sample gene expression profile of at least said genes; and
b) making a prediction of the prognosis of the patient based on the sample gene expression profile.

For example, the gene expression of at least 35, 40, 45, 50, 60, 70, 80, 90 or 100 genes may be measured. The at least 30 genes may include any or all of:
(a) A1CF, ACVR1B, ADAM28, ADM, ALDH1A1, ANGPTL3, APOH, ARRDC4, BTC, Cl9orf77, C20orf46, CEACAM1, CELA1, CHST1, CLDN10, CLPS, COL8A1, CPA1, CPA2, CRYBA2, CTRC, CTRL, CYP4F3, DAPL1, EGFR, EGLN3, ELSPBP1, ENTPD3, ERBB3, F10, F7, FKBP11, GATM, GCGR, GLP1R, GLS, GP2, GRM5, HAO1, HSD11B2, IL20RA, INS, KLK4, LOXL4, LRAT, MAFB, MASP2, MIA2, MNX1, MOBKL1A, MX2, NUPR1, P2RX1, PDGFC, PDIA2, PEMT, PFKFB2, PFKFB3, PLIN3, PMEPA1, PNLIP, PNLIPRP1, PNLIPRP2, PRLR, RARRES2, RBP4, REG1B, ROBO3, SCD5, SERPINA1, SERPINA3, SERPIND1, SERPINI2, SH3BP4, SLC16A3, SLC2A1, SLC30A2, SLC7A2, SLC7A8, SMARCA1, SMOC2, SSX2IP, STEAP3, SUSD5, TACSTD2, TBC1D24, TFF1, TGFBR3, TGIF1, TM4SF1, TM4SF4, TM4SF5, TMEM176B, TMEM181, TMEM90B, TMPRSS4, TSHZ3, USP29, VEGFC, WNT4;
(b) ALDH1A1, ANGPTL3, APOH, C19orf77, CEACAM1, CELA1, CLDN10, CLPS, CPA1, CPA2, CRYBA2, CTRC, CTRL, CYP4F3, DAPL1, EGLN3, ELSPBP1, ENTPD3, GCGR, GLP1R, GLS, GP2, GRM5, HAO1, INS, KLK4, LOXL4, MAFB, MASP2, MIA2, MOBKL1A, P2RX1, PDIA2, PNLIP, PNLIPRP1, PNLIPRP2, PRLR, RBP4, REG1B, SCD5, SERPINA1, SERPIND1, SERPINI2, SLC16A3, STEAP3, TFF1, TM4SF4, TM4SF5, TMPRSS4, USP29;
(c) ANGPTL3, APOH, C19orf77, CELA1, CLDN10, CLPS, CPA1, CPA2, CRYBA2, CTRC, CTRL, CYP4F3, EGLN3, ENTPD3, GCGR, GLP1R, GLS, GP2, GRM5, HAO1, INS, KLK4, LOXL4, MAFB, MASP2, MIA2, P2RX1, PDIA2, PNLIP, PNLIPRP1, PNLIPRP2, REG1B, SCD5, SERPINA1, SERPIND1, SERPINI2, STEAP3, TFF1, TMPRSS4, USP29;
(d) ANGPTL3, APOH, CLDN10, CLPS, CPA1, CPA2, CRYBA2, CTRC, CTRL, CYP4F3, EGLN3, GLP1R, GP2, GRM5, HAO1, INS, LOXL4, MASP2, P2RX1, PDIA2, PNLIP, PNLIPRP1, PNLIPRP2, REG1B, SERPINA1, SERPIND1, SERPINI2, STEAP3, TFF1, USP29
(e) ANGPTL3, APOH, CLDN10, CLPS, CPA1, CPA2, CTRC, CTRL, CYP4F3, GP2, GRM5, HAO1, INS, MASP2, PDIA2, PNLIP, PNLIPRP1, PNLIPRP2, SERPIND1, USP29,
(f) CPA1, CPA2, CTRL, CYP4F3, GLS, GRM5, HAO1, KLK4, MAFB, MASP2, MOBKL1A, PNLIPRP1, SERPIND1, STEAP3, USP29;
(g) CPA1, CPA2, CTRC, CTRL, GLS, GRM5, MASP2, MOBKL1A, PNLIPRP1, USP29;
(h) CPA1, CTRL, GLS, GEMS, MASP2, MOBKL1A, PNLIPRP1, USP29;
(i) CTRL, GLS, GRM5, MASP2, MOBKL1A, USP29;
(j) GLS, GRM5, MOBKL1A, USP29; or
(k) GLS, GRM5.

The steps of the prognostic methods may also be used in methods for predicting treatment response, methods for predicting overall survival (OS), methods for stratifying/classifying patients, methods for selecting a suitable treatment for a patient, methods for selecting patients for treatment and in computer-implemented methods.

Step b) making a prediction of the prognosis of the patient based on the sample gene expression profile may comprise the optional step of (i) normalising the measured expression level of each gene relative to the expression level of one or more housekeeping genes. Suitable housekeeping genes include one or more, for example 3 or more, 4 or more, 5 or more, 10 or more, 15 or more 20 or more, or substantially all, or about 30, or all of those listed in table 4.

Step b) making a prediction of the prognosis of the patient based on the sample gene expression profile may comprise the step of (ii) comparing the sample gene expression profile, optionally after the normalising step, with one or more reference centroids comprising:
a first reference centroid that represents the summarised gene expression of the measured genes in an ‘insulinoma-like’ type patient;
a second reference centroid that represents the summarised gene expression of the measured genes in an ‘intermediate’ type patient;
a third reference centroid that represents the summarised gene expression of the measured genes in a ‘metastasis-like-primary’ (MLP) type patient. According to this embodiment, the method may comprise the additional steps of:
c) classifying the sample gene expression profile as belonging to the insulinoma-like, intermediate or MLP group having the reference centroid to which it is most closely matched; and
d) providing a prognosis based on the classification made in step c).

The reference centroids may have been pre-determined and may be obtained by retrieval from a volatile or non-volatile computer memory or data store. In particular the sample gene expression profile may be compared to all three reference centroids.

Example reference centroids comprise one, two or all three of the centroids shown in table 3.

TABLE 3
Example centroids

| genes | Insulinoma-like | Intermediate | MLP |
|---|---|---|---|
| CEACAM1 | −2.619 | 0.5175 | 0.4646 |
| INS | 2.1656 | −0.5311 | −0.281 |
| PFKFB2 | 2.0939 | −0.481 | −0.3042 |
| ELSPBP1 | 2.087 | −0.3975 | −0.3851 |
| MIA2 | −2.0783 | 0.6246 | 0.1547 |
| ENTPD3 | 2.0695 | −0.3349 | −0.4412 |
| GRM5 | 1.9661 | −0.4081 | −0.3292 |
| STEAP3 | 1.8861 | −0.6741 | −0.0332 |
| APOH | −1.843 | 0.7066 | −0.0155 |
| SERPINA1 | −1.8421 | 0.6017 | 0.0891 |
| A1CF | −1.8091 | 0.4938 | 0.1846 |
| PRLR | −1.7938 | 0.4453 | 0.2274 |
| F10 | −1.7023 | 0.6704 | −0.032 |
| TMEM176B | −1.6658 | 0.3388 | 0.2859 |
| MASP2 | 1.6557 | −0.4494 | −0.1715 |
| RBP4 | 1.5705 | −0.7774 | 0.1884 |
| CYP4F3 | −1.543 | 0.4915 | 0.0871 |
| CHST8 | 1.5392 | −0.2847 | −0.2925 |
| KLK4 | 1.5317 | −0.4333 | −0.1411 |
| USP29 | 1.5013 | −0.3892 | −0.1737 |
| CELA1 | 1.4676 | −0.5537 | 0.0033 |
| TM4SF4 | −1.4098 | 0.2599 | 0.2687 |
| TMPRSS4 | 1.3881 | −0.4395 | −0.0811 |
| SCD5 | 1.3817 | −0.3667 | −0.1515 |
| TM4SF5 | −1.3527 | 0.151 | 0.3563 |
| SERPIND1 | −1.2469 | 0.5658 | −0.0982 |
| P2RX1 | 1.2378 | −0.567 | 0.1028 |
| GLP1R | 1.227 | −0.7076 | 0.2475 |
| LRAT | −1.2001 | 0.3925 | 0.0576 |
| CASR | 1.1903 | −0.4101 | −0.0363 |
| DAPL1 | 1.1772 | −0.394 | −0.0474 |
| ERBB3 | −1.1551 | 0.2507 | 0.1824 |
| C19orf77 | −1.1366 | 0.5365 | −0.1103 |
| F7 | −1.1088 | 0.4146 | 0.0012 |
| PLIN3 | −1.1061 | 0.3651 | 0.0496 |
| NEFM | 1.0914 | −0.4468 | 0.0375 |
| MNX1 | 1.0502 | −0.187 | −0.2068 |
| ROBO3 | 1.0498 | −0.4796 | 0.0859 |
| CPA1 | 1.0396 | −0.171 | −0.2189 |
| CTRL | 1.0324 | −0.2598 | −0.1274 |
| TGFBR3 | 1.0314 | −0.3271 | −0.0597 |
| PNLIPRP2 | 1.0293 | −0.3144 | −0.0716 |
| TSHZ3 | 0.9894 | −0.5562 | 0.1852 |
| ADAMTS2 | 0.9775 | −0.1468 | −0.2198 |
| GLRA2 | −0.9719 | 0.444 | −0.0796 |
| HGD | 0.9546 | 0.1951 | 0.1629 |
| GP2 | 0.9486 | −0.1884 | −0.1674 |
| CTRC | 0.9472 | −0.1359 | −0.2193 |
| RAB17 | −0.943 | 0.1644 | 0.1892 |
| ANGPTL3 | −0.9309 | 0.7313 | −0.3822 |
| LOXL4 | −0.9227 | 0.8894 | −0.5434 |
| PNLIP | 0.9217 | −0.1173 | −0.2283 |
| PEMT | −0.9181 | 0.1348 | 0.2094 |
| CPA2 | 0.898 | −0.1357 | −0.201 |
| PNLIPRP1 | 0.89 | −0.2451 | −0.0887 |
| ALDH1A1 | −0.888 | 0.4516 | −0.1186 |
| SLC12A7 | −0.8633 | 0.048 | 0.2757 |
| IL20RA | 0.8596 | −0.6899 | 0.3675 |
| CLPS | 0.8537 | −0.0882 | −0.232 |
| GLS | −0.8338 | 0.6425 | −0.3299 |
| C20orf46 | −0.8229 | 0.0879 | 0.2207 |
| GCGR | 0.8167 | −0.3211 | 0.0149 |
| IL18R1 | −0.8071 | 0.3806 | −0.078 |
| PDIA2 | 0.8067 | −0.2371 | −0.0655 |
| NAAA | −0.801 | 0.0699 | 0.2304 |
| BTC | −0.777 | 0.3415 | −0.0501 |
| TAPBPL | −0.7718 | 0.1346 | 0.1548 |
| ELMO1 | 0.7599 | −0.1868 | −0.0982 |
| KLK8 | −0.7466 | 0.3572 | −0.0772 |
| CDS1 | −0.7344 | 0.1808 | 0.0946 |
| TFF1 | −0.4502 | −0.5565 | 0.7253 |
| TBC1D24 | 0.7087 | −0.2012 | −0.0646 |
| KIT | −0.1886 | −0.6275 | 0.6983 |
| MOBKL1A | −0.6906 | 0.5167 | −0.2577 |
| PLA1A | −0.6807 | 0.0925 | 0.1627 |
| SUSD5 | 0.6571 | −0.4075 | 0.1611 |
| CRYBA2 | 0.0085 | 0.6535 | −0.6567 |
| PMM1 | −0.6512 | 0.129 | 0.1152 |
| EFNA1 | −0.6482 | −0.0629 | 0.3059 |
| SLC16A3 | −0.3093 | −0.5288 | 0.6448 |
| FKBP11 | −0.6405 | 0.2467 | −0.0065 |
| IL22RA1 | 0.0157 | −0.6362 | 0.6303 |
| ADM | −0.4275 | −0.4641 | 0.6244 |
| EGLN3 | −0.622 | −0.3749 | 0.6082 |
| LGALS4 | 0.2964 | −0.6215 | 0.5104 |
| TLE2 | −0.6031 | 0.2808 | −0.0546 |
| CLDN10 | 0.6022 | −0.2928 | 0.067 |
| NUPR1 | −0.0905 | −0.5664 | 0.6003 |
| SERPINI2 | 0.599 | −0.2985 | 0.0739 |
| PTPLA | −0.5914 | 0.1826 | 0.0392 |
| PVRL4 | 0.5913 | −0.4074 | 0.1857 |
| EGFR | −0.5301 | −0.3817 | 0.5805 |
| MAFB | 0.5783 | 0.2629 | −0.4798 |
| PFKFB3 | −0.2536 | −0.4824 | 0.5775 |
| HSD11B2 | 0.4836 | −0.5774 | 0.396 |
| FGB | −0.5585 | 0.1894 | 0.02 |
| NDC80 | −0.5544 | −0.3437 | 0.5517 |
| SMOC2 | 0.0794 | −0.5528 | 0.523 |
| ACVR1B | 0.4536 | −0.5522 | 0.3821 |
| TGIF1 | 0.2595 | −0.5502 | 0.4529 |
| ARRDC4 | −0.5175 | 0.4019 | −0.2078 |
| MMP1 | 0.2828 | −0.5127 | 0.4066 |
| TACSTD2 | 0.5006 | −0.4165 | 0.2288 |
| TOP2A | 0.2935 | −0.492 | 0.3819 |
| SH3BP4 | −0.0613 | −0.4678 | 0.4908 |
| PDGFC | 0.1177 | −0.4879 | 0.4437 |
| THBS2 | −0.2884 | −0.3781 | 0.4863 |
| CNPY2 | −0.4827 | 0.0704 | 0.1106 |
| HAO1 | −0.1631 | 0.4717 | −0.4105 |
| ADAM28 | 0.0504 | −0.4669 | 0.448 |
| C7orf68 | −0.4065 | −0.312 | 0.4644 |
| GATM | 0.4616 | −0.3139 | 0.1408 |
| CXCR4 | −0.1765 | −0.3947 | 0.4609 |
| PAFAH1B3 | −0.4603 | 0.0567 | 0.1159 |
| NEK6 | −0.4529 | −0.2507 | 0.4205 |
| AKR1C4 | −0.2208 | −0.3692 | 0.452 |
| F12 | −0.4515 | −0.1248 | 0.2941 |
| PMEPA1 | 0.449 | −0.4494 | 0.281 |
| RAB7L1 | 0.4491 | 0.0954 | −0.2638 |
| SMO | −0.0939 | −0.4117 | 0.4469 |
| CLDN1 | −0.4422 | 0.0249 | 0.1409 |
| CHST1 | 0.4421 | −0.3476 | 0.1818 |
| WNT4 | −0.231 | 0.4383 | −0.3517 |
| TMPRSS15 | −0.2167 | −0.3553 | 0.4365 |
| SPAG4 | −0.4348 | −0.1291 | 0.2921 |
| MX2 | −0.0034 | −0.4324 | 0.4337 |
| SLC7A2 | −0.076 | 0.4293 | −0.4008 |
| GUCA1C | −0.4275 | 0.2248 | −0.0645 |
| SLC7A8 | 0.4251 | 0.1764 | −0.3358 |
| PRSS22 | 0.4232 | −0.2329 | 0.0742 |
| RARRES2 | 0.1893 | −0.42 | 0.349 |
| PRSS8 | −0.4163 | 0.1247 | 0.0315 |
| SLC30A2 | 0.2978 | −0.4142 | 0.3025 |
| TMEM90B | −0.0705 | 0.4091 | −0.3827 |
| VIPR2 | 0.2079 | −0.4031 | 0.3251 |
| CXCR7 | −0.0836 | −0.3682 | 0.3996 |
| SMARCA1 | −0.3969 | 0.3089 | −0.1601 |
| FAM19A5 | −0.0086 | −0.3846 | 0.3878 |
| CLDN11 | 0.3874 | −0.0013 | −0.144 |
| SERPINA3 | 0.2386 | −0.3838 | 0.2944 |
| GAL3ST4 | −0.3788 | 0.0897 | 0.0523 |
| AFG3L1 | −0.376 | 0.1502 | −0.0092 |
| COL8A1 | −0.0067 | −0.3662 | 0.3687 |
| SSX2IP | −0.3254 | 0.368 | −0.2459 |
| IMPA2 | −0.2547 | −0.2701 | 0.3656 |
| VEGFC | −0.2604 | 0.3522 | −0.2546 |
| TMEM181 | 0.3434 | −0.2532 | 0.1245 |
| LGALS2 | 0.2734 | −0.3411 | 0.2386 |
| PLXDC1 | −0.1591 | −0.2811 | 0.3408 |
| TLR3 | 0.0666 | −0.3357 | 0.3108 |
| PSMB9 | −0.2906 | −0.2264 | 0.3354 |
| CHI3L2 | 0.3323 | −0.2335 | 0.1089 |
| PLCE1 | 0.3321 | −0.0457 | −0.0788 |
| ABI3BP | −0.3227 | 0.0663 | 0.0547 |
| NUDT5 | 0.3208 | −0.0512 | −0.0691 |
| FOXO4 | −0.3167 | −0.146 | 0.2647 |
| SLC2A1 | −0.149 | −0.2605 | 0.3164 |
| COL1A2 | 0.052 | −0.3153 | 0.2958 |
| REG1B | 0.3082 | −0.1317 | 0.0162 |
| NETO2 | −0.2815 | −0.2013 | 0.3069 |
| ENC1 | −0.1294 | −0.2538 | 0.3023 |
| DLL1 | −0.2356 | −0.1945 | 0.2829 |
| TM4SF1 | 0.0249 | −0.2812 | 0.2718 |
| CKS2 | 0.0047 | −0.2754 | 0.2737 |
| FGD1 | −0.2749 | −0.0247 | 0.1278 |
| PPEF1 | −0.2541 | −0.1781 | 0.2734 |
| LEF1 | −0.1015 | −0.2324 | 0.2704 |
| MLN | 0.1306 | −0.2663 | 0.2173 |
| TNFAIP6 | −0.2658 | −0.1274 | 0.2271 |
| ACAD9 | 0.2533 | −0.1142 | 0.0192 |
| TYMS | −0.2394 | −0.1627 | 0.2525 |
| ZNF521 | −0.2491 | 0.0771 | 0.0163 |
| ACADSB | 0.2474 | −0.1114 | 0.0187 |
| TSC2 | 0.2426 | 0.0098 | −0.1008 |
| HR | 0.0515 | −0.2371 | 0.2178 |
| DEFB1 | −0.0916 | −0.1918 | 0.2262 |
| GRSF1 | −0.1592 | 0.2219 | −0.1622 |
| ACE | −0.2182 | 0.0208 | 0.061 |
| SRGAP3 | 0.2144 | −0.072 | −0.0084 |
| SMEK1 | −0.2144 | 0.0146 | 0.0658 |
| TWIST1 | −0.0591 | −0.1706 | 0.1928 |
| FMNL1 | 0.1916 | −0.1785 | 0.1067 |
| ADAMTS7 | −0.1902 | 0.0895 | −0.0182 |
| COL5A2 | 0.118 | −0.1878 | 0.1435 |
| IFI44 | −0.175 | −0.0689 | 0.1345 |
| CAPN13 | 0.0494 | −0.1671 | 0.1486 |
| AQP8 | 0.1354 | 0.1002 | −0.151 |
| IP6K2 | 0.1456 | −0.0236 | −0.031 |
| COPE | −0.1402 | 0.0235 | 0.0291 |
| MXRA5 | −0.1284 | −0.0335 | 0.0817 |
| RBPJL | 0.019 | 0.1183 | −0.1255 |
| MBP | −0.0392 | −0.1016 | 0.1163 |
| MAP3K14 | 0.0979 | −0.1025 | 0.0658 |
| CLCA1 | 0.0703 | −0.0936 | 0.0672 |
| IDS | 0.0688 | 0.0215 | −0.0473 |
| TECR | 0.0606 | 0.0193 | −0.042 |
| CAPNS1 | −0.0055 | −0.0539 | 0.0559 |
| POSTN | −0.0558 | 0.0271 | −0.0062 |

It has historically been difficult to identify which patients are at high risk of, or are likely to have tumours which metastasize. Information such as this is valuable in determining a preferred treatment plan for a patient. According to the present invention MLP type PanNETs are more like to metastasize that other PanNETs. Accordingly, patients having MLP type PanNETs may be identified as being at high risk of metastasis. Such patients may be selected from treatments in line with patients at high risk of poor prognosis.

The insulinoma-like type group is indicative of a good prognosis. Accordingly, when the sample gene expression profile is classified as ‘insulinoma-like’ type, the step (d) of providing a prediction of prognosis may comprise prediction of a good prognosis. In other words, when the sample gene expression profile is classified as insulinoma-like, the patient is at low risk of poor prognosis.

Likewise, the intermediate type group is indicative of a good prognosis. Accordingly, when the sample gene expression profile is classified as ‘intermediate’ type, the step (d) of providing a prediction of prognosis may comprise prediction of a good prognosis. In other words, when the sample gene expression profile is classified as intermediate, the patient is at low risk of poor prognosis.

The MLP type groups is indicative of a poor prognosis. Accordingly, when the sample gene expression profile is classified as ‘MLP’ type, the step (d) of providing a prediction of prognosis may comprise prediction of a poor prognosis. In other words, when the sample gene expression profile is classified as MLP, the patient is at high risk of poor prognosis.

Alternatively, in addition to the optional normalising step (i) described above, step b) making a prediction of the prognosis of the patient based on the sample gene expression profile may comprise (ii) comparing the sample gene expression profile, optionally after the normalising step (i), with the expression profile of:
a high risk control group of PanNET patients known to have had a median overall survival time post-diagnosis of less than 71 months, or even less than 60 months; and
a low risk control group of PanNET patients known to have had a median overall survival time post-diagnosis of greater than 71 months, or even more than 100 months.

These methods may comprise the additional steps of:
c) classifying the sample gene expression profile as belonging to the risk group having the gene expression profile to which it is most closely matched; and
d) providing a prediction of prognosis based on the classification made in step c).

In this method, step (ii) of comparing the sample gene expression profile with the expression profiles of a high risk and a low risk control group, may comprise comparing the sample gene expression profile with reference centroids that corresponding to the low and high risk subgroups, respectively. In this instance the reference centroid would comprise:
a first reference centroid that represents the summarised gene expression of the high risk patients measured in a high risk training set made up of PanNET patients known to have had a median overall survival time post-diagnosis of less than 71 months, or even less than 60 months;
a second reference centroid that represents the summarised gene expression of the low risk patients measured in a low risk training set made up of PanNET patients known to have had a median overall survival time post-diagnosis of greater than 71 months, or even more than 100 months.

According to any of the methods of involving comparison of a sample gene expression profile with a reference centroid, Pearsons correlation may be used to make this comparison with each reference centroid for closeness of fit. The reference centroids may have been pre-determined and may be obtained by retrieval from a volatile or non-volatile computer memory or data store.

In addition to the gene expression profiles as discussed above, the methods may comprise the additional step of identifying any mutations within one of more of the genes selected from: MEN1, ATRX, DAXX, PTEN, TSC1, TSC2 and ATM in a sample obtained from the PanNET of the patient, wherein step (b) involves making a prediction of the prognosis of the patient based on the sample gene expression profile and the mutation status of the one or more genes.

The investigation of the mutation status of these genes, and use of them as biomarkers may increase the predictive prognostic value.

In particular 2, 3, 4, 5, 6 or all of these genes are investigated for mutations. In particular, MEN1 may be investigated for mutations. All of the genes may be investigated for mutations.

The enrichment of mutations in one or more of these genes may be used to further classify the sub-type of PanNET. For example, the mutation status may be used to inform selection of therapy. For example, the presence of a (one or more) mutations, in particular the enrichment of mutations, in a gene may result in selection of a drug that targets that gene.

For example, if there are (one or more) mutations, in particular enrichment of mutations, in ATM, the patient may be identified or selected for treatment with a PARP inhibitor (Choi et al. ATM Mutations in Cancer: Therapeutic Implications Mol Cancer Ther Aug. 1 2016 (15) (8) 1781-1791; Wang et al. ATM-Deficient Colorectal Cancer Cells Are Sensitive to the PARP Inhibitor Olaparib. Transl Oncol. 2017 April; 10(2):190-196. doi: 10.1016).

For example, if there are (one or more) mutations, in particular enrichment of mutations, in PTEN, TSC1 and/or TSC2, the patient may be identified or selected for treatment with an mTOR inhibitor, e.g. everolimus (Owonikoko and Khuri, Targeting the PI3K/AKT/mTOR Pathway: Biomarkers of Success and Tribulation Am Soc Clin Oncol Educ Book. 2013: 10.1200).

Other therapies based on mutations in these genes are available.

In some embodiments, the methods comprise the additional step of administering a therapy (e.g. a PARP inhibitor) to the patient identified or selected for that treatment.

The presence of (one or more) mutations, in particular the enrichment of mutations, in MEN1 is indicative of the patient being an intermediate subtype patient. The presence of a mutation in MEN1 may be indicative of good prognosis.

Accordingly, for a PanNET having a MEN1 mutation, the method may include the step of providing a prediction of good prognosis. For a PanNET having a MEN1 mutation, the patient may be determined to be at low risk of poor prognosis. In particular, where the gene expression profile is classified as ‘intermediate’ type and the presence of a mutation in MEN1 is identified, the method may include the step of providing a prediction of good prognosis, or identifying the patient as at low risk of poor prognosis.

The presence of a (one or more) mutations, in particular the enrichment of mutations, in DAXX and/or ATRX is indicative of the PanNET being an intermediate subtype or MLP subtype.

The presence of a (one or more) mutations, in particular the enrichment of mutations, in TSC2, PTEN and/or ATM is indicative of the PanNET being an intermediate subtype or MLP subtype.

According to the methods, a patient, having been determined to be at high risk of poor prognosis, or having been predicted to have a poor prognosis, may be selected for additional or alternative treatment, including aggressive treatment. For example, such ‘high risk’ patients may be treated with platinum-based chemotherapy doublets. These patients may be selected for therapeutic trials. Such patients may be selected for treatment with one or more of: platinum-based chemotherapy doublets, sunitinib, everolimus, peptide receptor radionuclide therapy (PRRT), chemotherapy and therapeutic trials. Such patients may be de-selected from non-treatment and monitoring.

A patient, having been found to be at low risk of poor prognosis, or having been predicted to have a good prognosis may be selected for less aggressive ongoing treatment or for monitoring or non-treatment. Such ‘low risk’ patients may be treated with surgery and/or somatostatin analogues, or the PanNET may be monitored. In other words, such patients may be selected for non-treatment and monitoring, or treatment with somatostatin analogues (e.g. octreotide).

Other factors, such as the stage of the disease as well as functionality and burden of metastatic disease, may be taken into account when selecting the therapy.

As discussed above, the PanNET subtypes identified herein provide a predictor of overall survival independent from the grade system previously used. Accordingly in some embodiments the methods of patient stratification or predicting the prognosis of a human pancreatic neuroendocrine tumor (PanNET) patient may be used as a stand-alone method.

The methods may also be used alongside other methods to help further classify patients. For example one or more of: the grade, the stage of the disease, functionality and burden of metastatic disease, may be taken into account when classifying patients, predicting prognosis, and selecting treatment options.

More grade-3 PanNETs are in the MLP subtype, and are associated with poor prognosis. These data suggest that subtyping using the methods described herein can facilitate patient stratification, potentially being able to identify patients having grade 1/2 PanNETs, whose disease may behave more aggressively than would be expected according to grade alone.

Accordingly, in some embodiments of the methods the PanNET in the patient has already been classified as grade 1/2 according to the WHO classification system, in particular according to the 2010 or 2017 WHO GEP-NET classification system, referred to elsewhere herein.

In some embodiments the methods of predicting the prognosis of a human pancreatic neuroendocrine tumor (PanNET) patient described herein may be used alongside the grade system. According to such uses, the methods may be used to further identify grade 1/2 patients that have MLP type PanNETs, as at high risk of poor prognosis, or predicting a poor prognosis.

Such patients may have a PanNET that may behave more aggressively than would be expected according to grade alone. Accordingly, such patients may be treated with earlier therapy with targeted treatment (e.g. sunitinib/everolimus) or PRRT or chemotherapy rather than ‘watchful waiting’ (non-treatment and monitoring) or just somatostatin analogues.

According to such methods, PanNETs identified as grade 1/2 may be further classified according to the methods described herein as belonging to a high risk group, or MLP group. In this case the patient is identified as at high risk of poor prognosis, or is predicted to have a poor prognosis. Such patients may be treated as high risk/poor prognosis patients as described elsewhere herein.

Similarly, in some embodiments of the methods, the PanNet may have already been classified as grade 3 according to the WHO classification system, in particular according to the 2010 or 2017 WHO GEP-NET classification system, referred to elsewhere herein.

The methods may be used to further identify grade 3 patients that have insulinoma-like or intermediate type PanNETs as at low risk of poor prognosis, or predicting a good prognosis.

According to such methods, PanNETs identified as grade 3 may be further classified according to the methods described herein as belonging to a low risk group, or intermediate or insulinoma-like group. In this case the patient is identified as at low risk of poor prognosis, or is predicted to have a good prognosis. Such patients may be treated as low risk/good prognosis patients as described elsewhere herein.

Although the methods and steps described above are largely in the context of predicting the prognosis of a human pancreatic neuroendocrine tumor patient, the steps and features described herein can also be used in computer implemented methods, and methods of treatment.

For example, the invention comprises a computer-implemented method for predicting the prognosis of a human PanNET patient, the method comprising:
a) obtaining gene expression data comprising a gene expression profile representing gene expression measurements of at least 30 genes selected from:
CEACAM1, INS, PFKFB2, ELSPBP1, MIA2, ENTPD3, GRM5, STEAP3, APOH, SERPINA1, A1CF, PRLR, F10, TMEM176B, MASP2, RBP4, CYP4F3, CHST8, KLK4, USP29, CELA1, TM4SF4, TMPRSS4, SCD5, TM4SF5, SERPIND1, P2RX1, GLP1R, LRAT, CASR, DAPL1, ERBB3, C19orf77, F7, PLIN3, NEFM, MNX1, ROBO3, CPA1, CTRL, TGFBR3, PNLIPRP2, TSHZ3, ADAMTS2, GLRA2, HGD, GP2, CTRC, RAB17, ANGPTL3, LOXL4, PNLIP, PEMT, CPA2, PNLIPRP1, ALDH1A1, SLC12A7, IL20RA, CLPS, GLS, C20orf46, GCGR, IL18R1, PDIA2, NAAA, BTC, TAPBPL, ELMO1, KLK8, CDS1, TFF1, TBC1D24, KIT, MOBKL1A, PLA1A, SUSD5, CRYBA2, PMM1, EFNA1, SLC16A3, FKBP11, IL22RA1, ADM, EGLN3, LGALS4, TLE2, CLDN10, NUPR1, SERPINI2, PTPLA, PVRL4, EGFR, MAFB, PFKFB3, HSD11B2, FGB, NDC80, SMOC2, ACVR1B, TGIF1, ARRDC4, MMP1, TACSTD2, TOP2A, SH3BP4, PDGFC, THBS2, CNPY2, HAO1, ADAM28, C7orf68, GATM, CXCR4, PAFAH1B3, NEK6, AKR1C4, F12, PMEPA1, RAB7L1, SMO, CLDN1, CHST1, WNT4, TMPRSS15, SPAG4, MX2, SLC7A2, GUCA1C, SLC7A8, PRSS22, RARRES2, PRSS8, SLC30A2, TMEM90B, VIPR2, CXCR7, SMARCA1, FAM19A5, CLDN11, SERPINA3, GAL3ST4, AFG3L1, COL8A1, SSX2IP, IMPA2, VEGFC, TMEM181, LGALS2, PLXDC1, TLR3, PSMB9, CHI3L2, PLCE1, ABI3BP, NUDT5, FOXO4, SLC2A1, COL1A2, REG1B, NETO2, ENC1, DLL1, TM4SF1, CKS2, FGD1, PPEF1, LEF1, MLN, TNFAIP6, ACAD9, TYMS, ZNF521, ACADSB, TSC2, HR, DEFB1, GRSF1, ACE, SRGAP3, SMEK1, TWIST1, FMNL1, ADAMTS7, COL5A2, IFI44, CAPN13, AQP8, IP6K2, COPE, MXRA5, RBPJL, MBP, MAP3K14, CLCA1, IDS, TECR, CAPNS1, POSTN, measured in a sample obtained from the PanNET of the patient; and
b) (i) optionally, normalising the measured expression level of each gene relative to the expression level of one or more housekeeping genes,
(ii) comparing the sample gene expression profile with two or three reference centroids as defined above (relating to high risk and low risk patients, or to insulinoma-like, intermediate and MLP type patinets);
c) classifying the sample gene expression profile as belonging to the group having the reference centroid to which it is most closely matched; and
d) providing a prediction of prognosis based on the classification made in step c).

As described above, the sample gene expression profile may be compared with each reference centroid for closeness of fit using Pearson correlation.

In addition the methods described may be described as methods of treatment or methods of selecting a patient for treatment. Accordingly, the method may include a step of selecting a patient for treatment using their predicted prognosis or identification as high/low risk. The method may comprise a step of administering the treatment to a patient in need thereof. The invention also provides agents for use in methods of treatment.

The invention provides a method of treatment of PanNET in a human patient, the method comprising:
(a) carrying out the methods as described herein; and
(b) (i) when the patient is determined to be at high risk of poor prognosis, or is predicted to have a poor prognosis, administering additional anti-tumor therapy or a more aggressive anti-tumor therapy; or
(ii) when the patient is determined to be at low risk of poor prognosis, or is predicted to have a good prognosis, not administering additional anti-tumor therapy or administering anti-tumor therapy that is less aggressive.

When the patient is determined to be at high risk of poor prognosis, or is predicted to have a poor prognosis, the patient may be selected for treatment with one or more of: platinum-based chemotherapy doublets, sunitinib, everolimus, peptide receptor radionuclide therapy (PRRT), chemotherapy and therapeutic trials as described elsewhere herein.

When the patient is determined to be at high risk of poor prognosis, or is predicted to have a poor prognosis, the patient may be selected for treatment with one or more of: sunitinib, everolimus, peptide receptor radionuclide therapy (PRRT), chemotherapy and therapeutic trials as described elsewhere herein. Such patients may be de-selected from non-treatment and monitoring. When the patient is determined to be at low risk of poor prognosis, or is predicted to have a good prognosis, the patient is selected for non-treatment and monitoring, or treatment by surgery and/or somatostatin analogues as described elsewhere herein.

The platinum-based chemotherapy doublets, somatostatin analogues, sunitinib, everolimus, and any other therapeutic agents are contemplated for use in methods of treatment of patients that have been classified according to the invention.

In accordance with any aspect of the present invention, the patient may be a human, particularly a human who has been diagnosed as having a pancreatic neuroendocrine tumor (PanNET). In some cases the patient may be a plurality of patients. In particular, the methods of the present invention may be for stratifying a group of patients (e.g. for a clinical trial) into high and low risk subgroups based on their gene expression profiles.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows the median OS according to subtype assigned by NanoString 228-Gene assay (n=106). Clinical data was available for 106 patients whose samples were assessed using the 228-gene (30 of them are housekeeping genes) NanoString assay. OS according to subtype is shown. Using Kaplan-Meier analysis the MLP patients had a significantly worse prognosis than the Insulinoma-like patients with a median OS of 71 months whereas OS was not reached for Insulinoma-like or Intermediate patients. (top line—Insulinoma; middle line—Intermediate; bottom line—MLP)

FIG. 2 shows median overall survival according to WHO Grade in patients selected for 228-Gene Nanostring Assay with clinical data available (n=106). Clinical data was available for 106 patients whose samples were assessed using the 228-gene NanoString assay. OS according to Grade is shown. Survival was associated with Grade of disease with Grade 3 patients having a significantly worse median OS of 24 months, consistent with published data. It should be noted that only 14% of the MLP patients analysed had Grade 3 disease, demonstrating the ability of the PanNETassigner NanoString assay to highlight those patients with Grade 1 and 2 disease who have a worse prognosis than may be expected according to the Grade alone. (bottom-left line—Grade 3; middle line—Grade 2; top-right line—Grade 1)

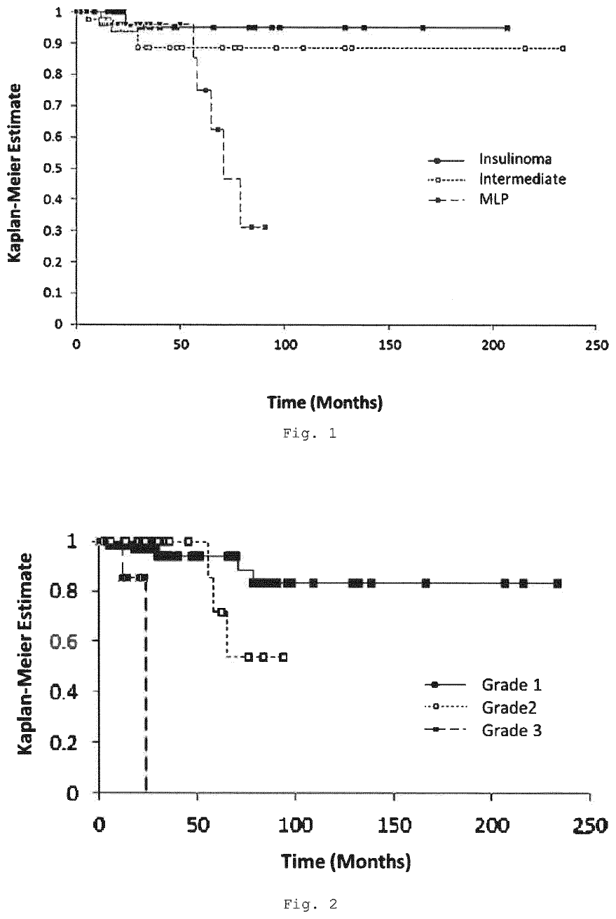

### DETAILED DESCRIPTION OF THE INVENTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Additionally, “A, B and/or C” is equivalent to “one or more of A, B and C”.

### Samples

A “sample” as used herein may be a cell or tissue sample (e.g. a biopsy), a biological fluid, an extract (e.g. a protein or DNA extract obtained from the subject). In particular, the sample may be a tumor sample, in particular a sample from the PanNET. The sample may be one which has been freshly obtained from the subject or may be one which has been processed and/or stored prior to making a determination (e.g. frozen, fixed or subjected to one or more purification, enrichment or extractions steps). For example, the sample may be fresh-frozen or formalin-fixed paraffin-embedded samples.

### Gene Expression

Reference to determining the expression level refers to determination of the expression level of an expression product of the gene. Expression level may be determined at the nucleic acid level or the protein level.

The gene expression levels determined may be considered to provide an expression profile. By “expression profile” is meant a set of data relating to the level of expression of one or more of the relevant genes in an individual, in a form which allows comparison with comparable expression profiles (e.g. from individuals for whom the prognosis is already known), in order to assist in the determination of prognosis and in the selection of suitable treatment for the individual patient.

The determination of gene expression levels may involve determining the presence or amount of mRNA in a sample of tumor cells. Methods for doing this are well known to the skilled person. Gene expression levels may be determined in a tumor sample using any conventional method, for example using nucleic acid microarrays or using nucleic acid synthesis (such as quantitative PCR). For example, gene expression levels may be determined using a NanoString nCounter Analysis system (see, e.g., U.S. Pat. No. 7,473,767).

Alternatively or additionally, the determination of gene expression levels may involve determining the protein levels expressed from the genes in a sample containing tumor cells obtained from an individual. Protein expression levels may be determined by any available means, including using immunological assays. For example, expression levels may be determined by immunohistochemistry (IHC), Western blotting, ELISA, immunoelectrophoresis, immunoprecipitation and immunostaining. Using any of these methods it is possible to determine the relative expression levels of any or all of proteins expressed from the genes listed in table 5.

Gene expression levels may be compared with the expression levels of the same genes in tumors from a group of patients whose survival time is known. The patients to which the comparison is made may be referred to as the ‘control group’. Accordingly, the determined gene expression levels may be compared to the expression levels in a control group of individuals having a PanNET. The comparison may be made to expression levels determined in tumor cells of the control group. The comparison may be made to expression levels determined in samples of tumor cells from the control group. The tumor in the control group is the same type of tumor (ie. PanNET) as in the individual.

Other factors may also be matched between the control group and the individual and tumor being tested. For example the stage of tumor may be the same, the subject and control group may be age-matched and/or gender matched.

Additionally the control group may have been treated with the same form of surgery and/or same therapeutic treatment.

Accordingly, an individual may be stratified or grouped according to their similarity of gene expression with a group with high risk of poor prognosis or low risk of poor prognosis.

### Methods for Classification Based on Gene Expression

The present invention provides methods for predicting treatment response, predicting prognosis, classifying, or monitoring PanNET in subjects. In particular, data obtained from analysis of gene expression may be evaluated using one or more pattern recognition algorithms.

Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a particular PanNET prognosis subgroup: high risk or moderate risk), and second to classify an unknown sample (e.g., “test sample”) according to subgroup.

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements.

There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm. Such unsupervised methods include non-negative matrix factorisation (NMF), and can be used as an initial step to identify subgroups.

The other approach is termed “supervised” whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models are sometimes termed “expert systems,” but may be based on a range of different mathematical procedures such as support vector machine, decision trees, k-nearest neighbour and naïve Bayes. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis. Such unsupervised methods include Prediction Analysis for Microarrays (PAM) and Significance Analysis of Microarrays (SAM).

After stratifying the training samples according to subtype, a centroid-based prediction algorithm may be used to construct centroids based on the expression profile of the gene set described in table 5.

“Translation” of the descriptor coordinate axes can be useful. Examples of such translation include normalization and mean-centering. “Normalization” may be used to remove sample-to-sample variation. Some commonly used methods for calculating normalization factor include: (i) global normalization that uses all genes on the microarray or nanostring codeset; (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (Suppl.), 496-501).

In one embodiment, the genes listed in table 5 can be normalized to one or more control housekeeping genes. Exemplary housekeeping genes include:

TABLE 4
Exemplary housekeeping genes

| gene | NCBI Accession |
|---|---|
| AGK | NM_018238.3 |
| AMMECR1L | NM_001199140.1 |
| CC2D1B | NM_032449.2 |
| CNOT10 | NM_001256741.1 |
| CNOT4 | NM_001190848.1 |
| COG7 | NM_153603.3 |
| DDX50 | NM_024045.1 |
| DHX16 | NM_001164239.1 |
| DNAJC14 | NM_032364.5 |
| EDC3 | NM_001142443.1 |
| EIF2B4 | NM_172195.3 |
| ERCC3 | NM_000122.1 |
| FCF1 | NM_015962.4 |
| GPATCH3 | NM_022078.2 |
| HDAC3 | NM_003883.2 |
| MRPS5 | NM_031902.3 |
| MTMR14 | NM_022485.3 |
| NOL7 | NM_016167.3 |
| NUBP1 | NM_001278506.1 |
| PRPF38A | NM_032864.3 |
| SAP130 | NM_024545.3 |
| SF3A3 | NM_006802.2 |
| TLK2 | NM_006852.2 |
| TMUB2 | NM_024107.2 |
| TRIM39 | NM_021253.3 |
| USP39 | NM_001256725.1 |
| ZC3H14 | NM_001160103.1 |
| ZKSCAN5 | NM_014569.3 |
| ZNF143 | NM_003442.5 |
| ZNF346 | NM_012279.3 |

The nucleotide sequence for each gene as disclosed at that reference on 16 Feb. 2018 is expressly incorporated herein by reference.

It will be understood by one of skill in the art that the methods disclosed herein are not bound by normalization to any particular housekeeping genes, and that any suitable housekeeping gene(s) known in the art can be used. Many normalization approaches are possible, and they can often be applied at any of several points in the analysis. In one embodiment, microarray data is normalized using the LOWESS method, which is a global locally weighted scatterplot smoothing normalization function. In another embodiment, qPCR and NanoString nCounter analysis data is normalized to the geometric mean of set of multiple housekeeping genes. nSolver™ software analysis system can be used for this purpose. qPCR can be analysed using the fold-change method.

“Mean-centering” may also be used to simplify interpretation for data visualisation and computation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

When comparing data from multiple analyses (e.g. comparing expression profiles for one or more test samples to the centroids constructed from samples collected and analyzed in an independent study), it will be necessary to normalize data across these data sets.

Distance Weighted Discrimination (DWD) may be used to combine these data sets together (Benito et al. (2004) Bioinformatics 20(1): 105-114, incorporated by reference herein in its entirety). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other.

Further methods for combining data sets include the Combat method and others described in Lagani et al., BMC Bioinformatics, 2016, Vol. 17 (Suppl 5): 290, the entire contents of which is expressly incorporated herein by reference. Combat is a method specifically devised for removing batch effects in gene-expression data (Johnson W E, Li C, Rabinovic A. Adjusting batch effects in microarray expression data using empirical Bayes methods. Biostatistics. 2007; 8:118-27, the entire contents of which is expressly incorporated herein by reference).

Clustering tools may be used to compare sample expression profiles to defined subtypes. Pearsons correlation may be used to compare sample expression profiles to defined subtypes.

The prognostic performance of the gene expression signature and/or other clinical parameters may assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., gene expression profile with or without additional clinical factors, as described herein). The “hazard ratio” is the risk of death at any given time point for patients displaying particular prognostic variables.

### Genes Making Up the Gene Signature or Gene Expression Profile

In accordance with any aspect of the present invention, the genes that make up the gene expression profile may be selected from 30 or more (such as all of the) genes selected from the following group: CEACAM1, INS, PFKFB2, ELSPBP1, MIA2, ENTPD3, GRM5, STEAP3, APOH, SERPINA1, A1CF, PRLR, F10, TMEM176B, MASP2, RBP4, CYP4F3, CHST8, KLK4, USP29, CELA1, TM4SF4, TMPRSS4, SCD5, TM4SF5, SERPIND1, P2RX1, GLP1R, LRAT, CASR, DAPL1, ERBB3, C19orf77, F7, PLIN3, NEFM, MNX1, ROBO3, CPA1, CTRL, TGFBR3, PNLIPRP2, TSHZ3, ADAMTS2, GLRA2, HGD, GP2, CTRC, RAB17, ANGPTL3, LOXL4, PNLIP, PEMT, CPA2, PNLIPRP1, ALDH1A1, SLC12A7, IL20RA, CLPS, GLS, C20orf46, GCGR, IL18R1, PDIA2, NAAA, BTC, TAPBPL, ELMO1, KLK8, CDS1, TFF1, TBC1D24, KIT, MOBKL1A, PLA1A, SUSD5, CRYBA2, PMM1, EFNA1, SLC16A3, FKBP11, IL22RA1, ADM, EGLN3, LGALS4, TLE2, CLDN10, NUPR1, SERPINI2, PTPLA, PVRL4, EGFR, MAFB, PFKFB3, HSD11B2, FGB, NDC80, SMOC2, ACVR1B, TGIF1, ARRDC4, MMP1, TACSTD2, TOP2A, SH3BP4, PDGFC, THBS2, CNPY2, HAO1, ADAM28, C7orf68, GATM, CXCR4, PAFAH1B3, NEK6, AKR1C4, F12, PMEPA1, RAB7L1, SMO, CLDN1, CHST1, WNT4, TMPRSS15, SPAG4, MX2, SLC7A2, GUCA1C, SLC7A8, PRSS22, RARRES2, PRSS8, SLC30A2, TMEM90B, VIPR2, CXCR7, SMARCA1, FAM19A5, CLDN11, SERPINA3, GAL3ST4, AFG3L1, COL8A1, SSX2IP, IMPA2, VEGFC, TMEM181, LGALS2, PLXDC1, TLR3, PSMB9, CHI3L2, PLCE1, ABI3BP, NUDT5, FOXO4, SLC2A1, COL1A2, REG1B, NETO2, ENC1, DLL1, TM4SF1, CKS2, FGD1, PPEF1, LEF1, MLN, TNFAIP6, ACAD9, TYMS, ZNF521, ACADSB, TSC2, HR, DEFB1, GRSF1, ACE, SRGAP3, SMEK1, TWIST1, FMNL1, ADAMTS7, COL5A2, IFI44, CAPN13, AQP8, IP6K2, COPE, MXRA5, RBPJL, MBP, MAP3K14, CLCA1, IDS, TECR, CAPNS1, POSTN.

TABLE 5
Gene list

| | | | | | |
|---|---|---|---|---|---|
| CEACAM1 | F7 | TAPBPL | TGIF1 | SLC30A2 | PPEF1 |
| INS | PLIN3 | ELMO1 | ARRDC4 | TMEM90B | LEF1 |
| PFKFB2 | NEFM | KLK8 | MMP1 | VIPR2 | MLN |
| ELSPBP1 | MNX1 | CDS1 | TACSTD2 | CXCR7 | TNFAIP6 |
| MIA2 | ROBO3 | TFF1 | TOP2A | SMARCA1 | ACAD9 |
| ENTPD3 | CPA1 | TBC1D24 | SH3BP4 | FAM19A5 | TYMS |
| GRM5 | CTRL | KIT | PDGFC | CLDN11 | ZNF521 |
| STEAP3 | TGFBR3 | MOBKL1A | THBS2 | SERPINA3 | ACADSB |
| APOH | PNLIPRP2 | PLA1A | CNPY2 | GAL3ST4 | TSC2 |
| SERPINA1 | TSHZ3 | SUSD5 | HAO1 | AFG3L1 | HR |
| A1CF | ADAMTS2 | CRYBA2 | ADAM28 | COL8A1 | DEFB1 |
| PRLR | GLRA2 | PMM1 | C7orf68 | SSX2IP | GRSF1 |
| F10 | HGD | EFNA1 | GATM | IMPA2 | ACE |
| TMEM176B | GP2 | SLC16A3 | CXCR4 | VEGFC | SRGAP3 |
| MASP2 | CTRC | FKBP11 | PAFAH1B3 | TMEM181 | SMEK1 |
| RBP4 | RAB17 | IL22RA1 | NEK6 | LGALS2 | TWIST1 |
| CYP4F3 | ANGPTL3 | ADM | AKR1C4 | PLXDC1 | FMNL1 |
| CHST8 | LOXL4 | EGLN3 | F12 | TLR3 | ADAMTS7 |
| KLK4 | PNLIP | LGALS4 | PMEPA1 | PSMB9 | COL5A2 |
| USP29 | PEMT | TLE2 | RAB7L1 | CHI3L2 | IFI44 |
| CELA1 | CPA2 | CLDN10 | SMO | PLCE1 | CAPN13 |
| TM4SF4 | PNLIPRP1 | NUPR1 | CLDN1 | ABI3BP | AQP8 |
| TMPRSS4 | ALDH1A1 | SERPINI2 | CHST1 | NUDT5 | IP6K2 |
| SCD5 | SLC12A7 | PTPLA | WNT4 | FOXO4 | COPE |
| TM4SF5 | IL20RA | PVRL4 | TMPRSS15 | SLC2A1 | MXRA5 |
| SERPIND1 | CLPS | EGFR | SPAG4 | COL1A2 | RBPJL |
| P2RX1 | GLS | MAFB | MX2 | REG1B | MBP |
| GLP1R | C20orf46 | PFKFB3 | SLC7A2 | NETO2 | MAP3K14 |
| LRAT | GCGR | HSD11B2 | GUCA1C | ENC1 | CLCA1 |
| CASR | IL18R1 | FGB | SLC7A8 | DLL1 | IDS |
| DAPL1 | PDIA2 | NDC80 | PRSS22 | TM4SF1 | TECR |
| ERBB3 | NAAA | SMOC2 | RARRES2 | CKS2 | CAPNS1 |
| C19orf77 | BTC | ACVR1B | PRSS8 | FGD1 | POSTN |

NCBI Accession numbers (Gene ID numbers) for these genes and the housekeeping genes are as indicated in brackets below: A1CF (NM_014576.2), ABI3BP (NM_015429.3), ACAD9 (NM_014049.4), ACADSB (NM_001609.3), ACE (NM_000789.2), ACVR1B (NM_004302.4), ADAM28 (NM_014265.4), ADAMTS2 (NM_021599.2), ADAMTS7 (NM_014272.3), ADM (NM_001124.1), AFG3L1 (NR 003228.1), AKR1C4 (NM_001818.2), ALDH1A1 (NM_000689.3), ANGPTL3 (NM_014495.2), APOH (NM_000042.2), AQP8 (NM_001169.2), ARRDC4 (NM_183376.2), BTC (NM_001729.2), C19orf77 (NM_001136503.1), C20orf46 (NM_018354.1), C7orf68 (NM_013332.1), CAPN13 (NM_144575.2), CAPNS1 (NM_001749.2), CASR (NM_000388.3), CDS1 (NM_001263.3), CEACAM1 (NM_001712.3), CELA1 (NM_001971.5), CHI3L2 (NM_004000.2), CHST1 (NM_003654.4), CHST8 (NM_001127895.1), CKS2 (NM_001827.1), CLCA1 (NM_001285.3), CLDN1 (NM_021101.3), CLDN10 (NM_001160100.1), CLDN11 (NM_001185056.1), CLPS (NM_001252598.1), CNPY2 (NM_001190991.1), COL1A2 (NM_000089.3), COL5A2 (NM_000393.3), COL8A1 (NM_001850.3), COPE (NM_199444.1), CPA1 (NM_001868.2), CPA2 (NM_001869.2), CRYBA2 (NM_057094.1), CTRC (NM_007272.2), CTRL (NM_001907.2), CXCR4 (NM_003467.2), CXCR7 (NM_020311.1), CYP4F3 (NM_000896.2), DAPL1 (NM_001017920.2), DEFB1 (NM_005218.3), DLL1 (NM_005618.3), EFNA1 (NM_004428.2), EGFR (NM_201282.1), EGLN3 (NM_022073.3), ELMO1 (NM_014800.9), ELSPBP1 (NM_022142.3), ENC1 (NM_003633.2), ENTPD3 (NM_001248.2), ERBB3 (NM_001005915.1), F10 (NM_000504.3), F12 (NM_000505.3), F7 (NM_019616.2), FAM19A5 (NM_015381.3), FGB (NM_005141.3), FGD1 (NM_004463.2), FKBP11 (NM_016594.2), FMNL1 (NM_005892.3), FOXO4 (NM_001170931.1), GAL3ST4 (NM_024637.4), GATM (NM_001482.2), GCGR (NM_000160.1), GLP1R (NM_002062.3), GLRA2 (NM_001118885.1), GLS (NM_014905.3), GP2 (NM_001502.2), GRM5 (NM_000842.1), GRSF1 (NM_001098477.1), GUCA1C (NM_005459.3), HAO1 (NM_017545.2), HGD (NM_000187.3), HR (NM_005144.4), HSD11B2 (NM_000196.3), IDS (NM_000202.6), IFI44 (NM_006417.4), IL18R1 (NM_003855.3), IL20RA (NM_014432.2), IL22RA1 (NM_021258.2), IMPA2 (NM_014214.2), INS (NM_000207.2), IP6K2 (NM_001005910.2), KIT (NM_000222.2), KLK4 (NM_004917.3), KLK8 (NM_144507.1), LEF1 (NM_016269.3), LGALS2 (NM_006498.2), LGALS4 (NM_006149.3), LOXL4 (NM_032211.6), LRAT (NM_004744.3), MAFB (NM_005461.3), MAP3K14 (NM_003954.1), MASP2 (NM_139208.1), MBP (NM_002385.2), MIA2 (NM_054024.3), MLN (NM_001184698.1), MMP1 (NM_002421.3), MNX1 (NM_005515.3), MOBKL1A (NM_173468.3), MX2 (NM_002463.1), MXRA5 (NM_015419.3), NAAA (NM_001042402.1), NDC80 (NM_006101.2), NEFM (NM_005382.2), NEK6 (NM_014397.3), NETO2 (NM_018092.3), NUDT5 (NM_014142.2), NUPR1 (NM_001042483.1), P2RX1 (NM_002558.2), PAFAH1B3 (NM_001145940.1), PDGFC (NM_016205.2), PDIA2 (NM_006849.2), PEMT (NM_148173.1), PFKFB2 (NM_001018053.1), PFKFB3 (NM_004566.3), PLA1A (NM_015900.2), PLCE1 (NM_001165979.1), PLIN3 (NM_001164194.1), PLXDC1 (NM_020405.4), PMEPA1 (NM_020182.3), PMM1 (NM_002676.2), PNLIP (NM_000936.2), PNLIPRP1 (NM_006229.2), PNLIPRP2 (NM_005396.4), POSTN (NM_001135935.1), PPEF1 (NM_006240.2), PRLR (NM_001204318.1), PRSS22 (NM_022119.3), PRSS8 (NM_002773.3), PSMB9 (NM_002800.4), PTPLA (NM_014241.3), PVRL4 (NM_030916.2), RAB17 (NR_033308.1), RAB7L1 (NM_001135664.1), RARRES2 (NM_002889.3), RBP4 (NM_006744.3), RBPJL (NM_001281449.1), REG1B (NM_006507.3), ROBO3 (NM_022370.2), SCD5 (NM_024906.2), SERPINA1 (NM_000295.4), SERPINA3 (NM_001085.4), SERPIND1 (NM_000185.3), SERPINI2 (NM_006217.3), SH3BP4 (NM_014521.2), SLC12A7 (NM_006598.2), SLC16A3 (NM_004207.2), SLC2A1 (NM_006516.2), SLC30A2 (NM_001004434.1), SLC7A2 (NM_001008539.3), SLC7A8 (NM_001267036.1), SMARCA1 (NM_003069.3), SMEK1 (NM_001284280.1), SMO (NM_005631.3), SMOC2 (NM_001166412.1), SPAG4 (NM_003116.1), SRGAP3 (NM_001033117.2), SSX2IP (NM_001166294.1), STEAP3 (NM_001008410.1), SUSD5 (NM_015551.1), TACSTD2 (NM_002353.2), TAPBPL (NM_018009.4), TBC1D24 (NM_020705.1), TECR (NR 038104.1), TFF1 (NM_003225.2), TGFBR3 (NM_003243.3), TGIF1 (NM_170695.2), THBS2 (NM_003247.2), TLE2 (NM_001144761.1), TLR3 (NM_003265.2), TM4SF1 (NM_014220.2), TM4SF4 (NM_004617.2), TM4SF5 (NM_003963.2), TMEM176B (NM_001101311.1), TMEM181 (NM_020823.1), TMEM90B (NM_024893.1), TMPRSS15 (NM_002772.2), TMPRSS4 (NM_019894.3), TNFAIP6 (NM_007115.2), TOP2A (NM_001067.3), TSC2 (NM_000548.3), TSHZ3 (NM_020856.2), TWIST1 (NM_000474.3), TYMS (NM_001071.1), USP29 (NM_020903.2), VEGFC (NM_005429.2), VIPR2 (NM_003382.3), WNT4 (NM_030761.3), ZNF521 (NM_015461.2), AGK (NM_018238.3), AMMECR1L (NM_031445.2), CC2D1B (NM_032449.2), CNOT10 (NM_001256741.1), CNOT4 (NM_001190848.1), COG7 (NM_153603.3), DDX50 (NM_024045.1), DHX16 (NM_001164239.1), DNAJC14 (NM_032364.5), EDC3 (NM_001142443.1), EIF2B4 (NM_172195.3), ERCC3 (NM_000122.1), FCF1 (NM_015962.4), GPATCH3 (NM_022078.2), HDAC3 (NM_003883.3), MRPS5 (NM_031902.3), MTMR14 (NM_022485.3), NOL7 (NM_016167.3), NUBP1 (NM_002484.3), PRPF38A (NM_032864.3), SAP130 (NM_024545.3), SF3A3 (NM_006802.2), TLK2 (XM_011524223.1), TMUB2 (NM_024107.2), TRIM39 (NM_021253.3), USP39 (NM_001256725.1), ZC3H14 (NM_207662.3), ZKSCAN5 (NM_014569.3), ZNF143 (NM_003442.5), ZNF346 (NM_012279.3).

The nucleotide sequence for each gene as disclosed at that accession number, on 16 Feb. 2018 is expressly incorporated herein by reference.

The expression levels of 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, or substantially all of, or all of the above genes (those listed in table 5) may be determined.

The inventors have shown that the use of at least 30 genes results in a misclassification error rate of around 0.04 (see table 13). It is noted that generally, larger numbers of genes are more likely to result in a more accurate (and useful) classification (see table 13). Accordingly, in some embodiments, at least 35, 40, 50, 60, 70, 80, 90, 100, 120 or more of the genes in table 5 are used in the methods of the invention.

In particular, the expression level of GLS may be determined as part of method step (a). In particular, the expression level of GRM5 may be determined as part of methods step (a).

The at least 30 genes may include any of the genes listed in the subgroups in table 13. For example, the at least 30 genes may include any or all of:
(a) A1CF, ACVR1B, ADAM28, ADM, ALDH1A1, ANGPTL3, APOH, ARRDC4, BTC, C19orf77, C20orf46, CEACAM1, CELA1, CHST1, CLDN10, CLPS, COL8A1, CPA1, CPA2, CRYBA2, CTRC, CTRL, CYP4F3, DAPL1, EGFR, EGLN3, ELSPBP1, ENTPD3, ERBB3, F10, F7, FKBP11, GATM, GCGR, GLP1R, GLS, GP2, GRM5, HAO1, HSD11B2, IL20RA, INS, KLK4, LOXL4, LRAT, MAFB, MASP2, MIA2, MNX1, MOBKL1A, MX2, NUPR1, P2RX1, PDGFC, PDIA2, PEMT, PFKFB2, PFKFB3, PLIN3, PMEPA1, PNLIP, PNLIPRP1, PNLIPRP2, PRLR, RARRES2, RBP4, REG1B, ROBO3, SCD5, SERPINA1, SERPINA3, SERPIND1, SERPINI2, SH3BP4, SLC16A3, SLC2A1, SLC30A2, SLC7A2, SLC7A8, SMARCA1, SMOC2, SSX2IP, STEAP3, SUSD5, TACSTD2, TBC1D24, TFF1, TGFBR3, TGIF1, TM4SF1, TM4SF4, TM4SF5, TMEM176B, TMEM181, TMEM90B, TMPRSS4, TSHZ3, USP29, VEGFC, WNT4;
(b) ALDH1A1, ANGPTL3, APOH, C19orf77, CEACAM1, CELA1, CLDN10, CLPS, CPA1, CPA2, CRYBA2, CTRC, CTRL, CYP4F3, DAPL1, EGLN3, ELSPBP1, ENTPD3, GCGR, GLP1R, GLS, GP2, GRM5, HAO1, INS, KLK4, LOXL4, MAFB, MASP2, MIA2, MOBKL1A, P2RX1, PDIA2, PNLIP, PNLIPRP1, PNLIPRP2, PRLR, RBP4, REG1B, SCD5, SERPINA1, SERPIND1, SERPINI2, SLC16A3, STEAP3, TFF1, TM4SF4, TM4SF5, TMPRSS4, USP29;
(c) ANGPTL3, APOH, C19orf77, CELA1, CLDN10, CLPS, CPA1, CPA2, CRYBA2, CTRC, CTRL, CYP4F3, EGLN3, ENTPD3, GCGR, GLP1R, GLS, GP2, GRM5, HAO1, INS, KLK4, LOXL4, MAFB, MASP2, MIA2, P2RX1, PDIA2, PNLIP, PNLIPRP1, PNLIPRP2, REG1B, SCD5, SERPINA1, SERPIND1, SERPINI2, STEAP3, TFF1, TMPRSS4, USP29;
(d) ANGPTL3, APOH, CLDN10, CLPS, CPA1, CPA2, CRYBA2, CTRC, CTRL, CYP4F3, EGLN3, GLP1R, GP2, GRM5, HAO1, INS, LOXL4, MASP2, P2RX1, PDIA2, PNLIP, PNLIPRP1, PNLIPRP2, REG1B, SERPINA1, SERPIND1, SERPINI2, STEAP3, TFF1, USP29
(e) ANGPTL3, APOH, CLDN10, CLPS, CPA1, CPA2, CTRC, CTRL, CYP4F3, GP2, GRM5, HAO1, INS, MASP2, PDIA2, PNLIP, PNLIPRP1, PNLIPRP2, SERPIND1, USP29,
(f) CPA1, CPA2, CTRL, CYP4F3, GLS, GRM5, HAO1KLK4, MAFB, MASP2, MOBKL1A, PNLIPRP1, SERPIND1, STEAP3, USP29;
(g) CPA1, CPA2, CTRC, CTRL, GLS, GRM5, MASP2, MOBKL1A, PNLIPRP1, USP29;
(h) CPA1, CTRL, GLS, GRM5, MASP2, MOBKL1A, PNLIPRP1, USP29;
(i) CTRL, GLS, GRM5, MASP2, MOBKL1A, USP29;
(j) GLS, GRM5, MOBKL1A, USP29; or
(k) GLS, GRM5.

### Additional Methods for Classification

In addition to the gene expression profiles for classifying, prognosticating, or monitoring PanNET in subjects, other biological markers, or ‘biomarkers’, can be used.

Accordingly, in some embodiments the methods of the invention comprising the additional steps of identifying any mutations within one or more of the genes: MEN1, STRX, DAXX, PTEN, TSC1, TSC2 and ATM. Mutations in the coding regions of these genes may be used to classify the PanNET.

In particular a (one or more) mutation, in particular the enrichment of mutations, in MEN1 is indicative of the patient being an intermediate subtype patient. A (one or more) mutation, in particular the enrichment of mutations, in DAXX and/or ATRX is indicative of the patient being an intermediate or MLP subtype patient. A (one or more) mutation, in particular the enrichment of mutations in TSC2, PTEN and/or ATM is indicative of the patient being an intermediate subtype or MLP subtype patient.

Mutations may be identified in the coding regions of genes using any method known in the art. For example DNA sequencing technology, for example Next Generation Sequencing (NGS), can be used to identify mutations. Examples of NGS techniques include methods employing sequencing by synthesis, sequencing by hybridisation, sequencing by ligation, pyrosequencing, nanopore sequencing, or electrochemical sequencing. Additional methods to detect the mutation include matrix-assisted laser desorption/ionization time-of-flight (MALDI-TOF) spectrometry, restriction fragment length polymorphism (RFLP), high-resolution melting (HRM) curve analysis, and denaturing high performance liquid Chromatography (DHPLC). Other PCR-based methods for detecting mutations include allele specific oligonucleotide polymerase chain reaction (ASO-PCR) and sequence-specific primer (SSP)-PCR. Mutations of may also be detected in mRNA transcripts through, for example, RNA sequence or reverse transcriptase PCR. Mutations may also be detected in the protein through, for example, peptide sequencing by mass spectrometry.

In this context, the mutations are as compared to the wild-type genes. In this context the wildtype genes are those provided at the NCBI accession numbers in table 6. Accordingly the mutations are not found in any of these wild-type genes. The mutations may be in the coding regions of the genes. The mutation(s) may result in deletions, substitutions, insertions, inversions, point-mutations, frame-shifting, or early truncation of the encoded protein. The mutations are non-synonymous.

TABLE 6

| gene | NCBI accession number |
|---|---|
| MEN1 | NM_000244.3 |
| | NM_130799.2 |
| | NM_130800.2 |
| | NM_130801.2 |
| | NM_130802.2 |
| | NM_130803.2 |
| | NM_130804.2 |
| ATRX | NM_000489.4 |
| | NM_138270.3 |
| DAXX | NM_001141969.1 |
| | NM_001141970.1 |
| | NM_001254717.1 |
| | NM_001350.4 |
| TSC1 | NM_000368.4 |
| | NM_001162426.1 |
| | NM_001162427.1 |
| TSC2 | NM_000548.4 |
| | NM_001077183.2 |
| | NM_001114382.2 |
| | NM_001318827.1 |
| | NM_001318829.1 |
| | NM_001318831.1 |
| | NM_001318832.1 |
| PTEN | NM_000314.6 |
| | NM_001304717.2 |
| | NM_001304718.1 |
| ATM | NM_000051.3 |
| | NM_001351834.1 |
| | NM_001351835.1 |
| | NM_001351836.1 |

### Prognosis

An individual grouped with the good prognosis group or low risk group, may be identified as being more likely to live longer.

In general terms, a “good prognosis” is one where survival (OS and/or PFS) of an individual patient can be favourably compared to what is expected in a population of patients within a comparable disease setting. This might be defined as better than median survival (i.e. survival that exceeds that of 50% of patients in population).

An individual grouped with the poor prognosis group or high risk group, may be identified as being less likely to live longer.

In general terms, a “poor prognosis” is one where survival (OS and/or PFS) of an individual patient can be unfavourably compared to what is expected in a population of patients within a comparable disease setting. This might be defined as worse than median survival (i.e. survival that exceeds that of 50% of patients in population).

Whether a prognosis is considered good or poor may vary between cancers and stage of disease. In general terms a good prognosis is one where the overall survival (OS) and/or progression-free survival (PFS) is longer than average for that stage and cancer type. A prognosis may be considered poor if PFS and/or OS is lower than average for that stage and type of cancer. The average may be the mean OS or PFS.

For example, a prognosis may be considered good if the OS is greater than 71 months from diagnosis. In particular, if the OS is greater than 100 or 120 months.

Similarly, OS of less than 71 months from diagnosis, in particular less than 60 months may be considered a poor prognosis.

As described in detail herein, the present inventors found that classification based on the gene expression model of the present invention was able to group patients into high risk and low risk subgroups. The median overall survival for high risk patients was 71 months and was not reached for low risk patients.

Accordingly a low risk control group of PanNET patients may be known to have had a median overall survival time post-diagnosis of greater than 71 months, or even more than 100 months, and a high risk control group of PanNET patients may be known to have had a median overall survival time post-diagnosis of less than 71 months, or even less than 60 months.

Where the individual is classified with the good prognosis/low risk group, the individual may be selected for treatment with suitable therapy as described in further detail below.

Where the individual is classified with the poor prognosis/high risk group, the individual may, for example, receive a novel or experimental therapy, or more aggressive therapy.

In embodiments of the invention in which the patients are classified into a subtype selected from MLP, Insulinoma and Intermediate, the classification as Insulinoma or Intermediate may be indicative of/predictive of a good prognosis or low risk of poor prognosis. The classification as MLP may be indicative of/predictive of a poor prognosis or high risk of poor prognosis.

### PanNET

As used herein “PanNET” refers to any pancreatic neuroendocrine tumor. It refers to sporadic tumors, and also includes secondary or metastatic tumors that have spread from the primary PanNET site in the pancreas to other sites.

### Therapy

There are several known therapies for PanNETs, which may be administered according to the subgroup of patient. Surgery may be used to treat all PanNet patients, or at least non-metastatic patients, with additional therapies applied based on subgroups.

For example, ‘high risk’ or MLP grouped patients, or patients predicted to have a poor prognosis according to the methods herein, may be treated in a similar manner to how grade 3 patients were treated. Such patients may be selected for aggressive therapy. For example these patients may be selected for treatment (and optionally treated) with platinum-based chemotherapy doublets, sunitinib, everolimus, peptide receptor radionuclide therapy (PRRT), and/or chemotherapy. These patients may also be selected for therapeutic trials. Such patients may be selected for treatment with combination therapies. Such patients may be selected for treatment with one or more of: platinum-based chemotherapy doublets, sunitinib, everolimus, peptide receptor radionuclide therapy (PRRT), chemotherapy, and therapeutic trials. Such treatments may be administered in addition to surgery and/or somatostatin analogues. Such patients may be de-selected from non-treatment and monitoring.

For example, ‘low risk’ or intermediate/insulinoma-like grouped patients, or patients predicted to have a good prognosis according to the methods herein, may be treated in a similar manner to how grade 1/2 patients were treated. Such patients may be selected for a less aggressive therapeutic approach. For example these patients may be treated with somatostatin analogues, optionally in addition to surgery, or the PanNet may be monitored but not treated. In other words, such patients may be selected for non-treatment and monitoring, or treatment by surgery and/or somatostatin analogues (e.g. octreotide).

The following is presented by way of example and is not to be construed as a limitation to the scope of the claims.

### EXAMPLES

### Materials and Methods

### Collection of PanNET Retrospective Samples

Verona Cohort

RNA isolated from fresh frozen tissue from patients undergoing resection of their primary PanNET disease was provided from 137 patients. A clinical database covering these patients was constructed.

### Nucleic Acid Extraction and Quality/Quantity Assessment

Following histopathologist assessment, selected tissue sections underwent deparaffinization, macrodissection and processing (RecoverAll™ Total Nucleic Acid Isolation Kit AM1975 protocol). Quality and quantity of extracted RNA was assessed using NanoDrop-2000 Spectrophotometer and Agilent RNA-6000 Bioanalyzer systems respectively. RNA was diluted for NanoString assay (100 ng/5 uL).

### NanoString Probe Development, Process and Analysis (PanNETassigner)

Probe Development

A panel of 228 genes (30 housekeeping), as shown in tables 4 and 3 respectively, was selected for a NanoString Elements™ assay based on our PanNETassigner signature²². Target specific probes were designed by NanoString. Probes were checked using Basic Local Alignment Search Tool (BLAST), an algorithm for comparing biological sequence information with established sequence databases, to confirm identity and optimum isoform coverage. Final probes were selected and ordered from Integrated DNA Technologies and TagSets from NanoString.

nCounter Elements™ Process

Oligonucleotide probe pools were created and hybridized to reporter/capture Tags, and these Tags were hybridized to the RNA target, according to the NanoString Elements™ manual (version 2, September 2016). Following hybridization, samples were purified, orientated and immobilised in their cartridge using the nCounter Prep Station before being loaded into the Digital Analyser. The molecular barcodes were counted and decoded, and the results stored as a Reporter Code Count (RCC) file. The RCC file was analysed alongside the Reporter Library File (RLF) containing details of the custom probes and housekeeping genes selected.

nSolver™ v3.0 analysis

The nSolver™ software analysis package was used to perform quality control (QC) and normalisation of the expression data. QC steps included assessment of assay metrics (field of view counts/binding density), internal CodeSet controls (6 positive, 8 negative controls to assess variations in expression level according to concentration and correct background noise respectively) and principal component analysis to assess batch effect. Following QC steps, raw data was normalised to housekeeping genes (those shown in table 4) selected using the geNorm algorithm within nSolver™.

### Assignment of Molecular Subtype and Refinement of 228-gene NanoString Assay

The normalised expression data was log2 transformed and median centred. PanNETassigner subtypes were assigned using Pearson correlation. The custom 228-gene NanoString assay was refined using unsupervised/supervised clustering methods and additional in-house developed bioinformatics techniques (iVLM).

Most methods available for integrative clustering assume that the underlying clustering structure is linear. However, clustering methods developed based on this assumption sometimes does not provide optimal results when the clustering structure is complex. Integrative latent variable model (iLVM) is a statistical tool developed to address this limitation capturing the dependence pattern between different omics data types to provide a global non-linear integrative clustering approach.

The key assumption governing iLVM framework is that features from different omics data types are correlated due to some “hidden” variables (meta-variables), which defines the underlying clustering structure between multiple omics data types. iLVM, simultaneously, projects all data types to a common low dimensional space (defined by the meta-variables), as well as assign samples into different clustering groups. In addition, the latent variables are allowed to be either common or data type specific in order to capture between and within data type variability.

The output of iLVM includes integrated subtypes and a panel of the most discriminative features spanning across different data types (possible biomarkers; genes, metabolites, peptides, etc.).

Standard nCounter Chemistry Process

The PanCancer Immune Profiling assay was ordered from NanoString Technologies. Hybridisation reactions were performed according to the nCounter® XT Assay Manual (Version 11, July 2016). The nCounter Prep Station, nCounter Digital Analyser and nSolver™ v3.0 analysis steps were carried out as above.

nCounter Advanced Analysis

Additional analysis was carried out using the nCounter Advanced Analysis Plugin, including immune cell type profiling and immune pathway scoring. Statistically significant differences between immune cell types profiled were assessed using Student's T-Test and corrected for multiple testing using Benjamini-Hochberg correction with a False Discovery Rate (FDR) of 0.05.

Development and Assignment of Immune Subtypes

Immune gene expression was across all samples, irrespective of PanNETassigner subtype, and according to PanNETassigner subtypes. Unsupervised (Non-negative Matrix Factorisation, NMF) and supervised (PAM/SAM) clustering methods were be used to develop specific immune subtypes.

Microarray

Microarray data available for PanNET samples from previous work conducted by The Institute of Cancer Research-Systems and Precision Cancer Medicine Team was used to validate the NanoString PanNETassigner signature work. Gene expression was assessed using Affymetrix GeneChip Human array and analysed using R and Bioconductor as previously described^{35,36}.

Targeted DNA sequencing

Human DNA samples were analysed with a panel testing of all known coding sequences for MEN1, ATRX, DAXX, PTAN, TSC2, MUTYH and ATM. NGS was performed as previously described³⁸.

### Example 1

### Developing the PanNET Gene Expression Assay

Developing the 228-gene NanoString Assay

An overview of the PanNET samples from the Verona cohort used for development and validation of the PanNET gene expression assay are shown in table 7. For the PanNET Verona Samples (n=222), the median RNA concentration was 222 ng/uL, range 2.8 to 4099 ng/uL. RNA Integrity number (RIN) ranged from 6.5 to 10.

TABLE 7
PanNET Verona Cohort

Matched Clinical Data n = 205
Fresh Frozen RNA provided from ARC-NET bio-bank n = 222
228 NanoString Gene Panel n = 144 (including 6 replicates and 7
matched normal samples)

The 228-gene assay was successfully developed as described in materials and methods. The assay was been performed on 144 samples from the Verona cohort including 6 replicates and 7 matched normal tissue. All samples passed QC as described in materials and methods. Heatmaps of the results for all samples and replicates were generated.

Validation of the 228-Gene NanoString Assay Results Using Microarray Data

Microarray data was available for n=19 PanNET samples analysed with the 228-gene NanoString assay. Concordance between subtypes assigned using microarray data and subtypes assigned using NanoString data was assessed in 2 ways; Pearson Correlation and integrative latent variable model (iLVM), a form of unsupervised clustering developed in-house.

The misclassification error rate was 5% using both methods (18/19 samples correctly classified) with a different sample misclassified using each method (Table 8).

TABLE 8

| | Microarray | NanoString Pearson | Nanostring |
|---|---|---|---|
| Sample | Subtype | Correlation Subtype | iLVM Subtype |
| 1634T | MLP | MLP | MLP |
| 1635T | MLP | MLP | MLP |
| 1637T* | MLP | Insulinoma | MLP |
| 1638T | Intermediate | Intermediate | Intermediate |
| 1644T | Insulinoma | Insulinoma | Insulinoma |
| 1649T | Intermediate | Intermediate | Intermediate |
| 1650T | Intermediate | Intermediate | Intermediate |
| 1656T | MLP | MLP | MLP |
| 1657T* | Intermediate | Intermediate | MLP |
| 1660T | MLP | MLP | MLP |
| 1665T | Intermediate | Intermediate | Intermediate |
| 1672T | Insulinoma | Insulinoma | Insulinoma |
| 1913T | MLP | MLP | MLP |
| 1914T | MLP | MLP | MLP |
| 1921T | Insulinoma | Insulinoma | Insulinoma |
| 1923T | Intermediate | Intermediate | Intermediate |
| 1929T | MLP | MLP | MLP |
| 1934T | Intermediate | Intermediate | Intermediate |
| 1935T | Intermediate | Intermediate | Intermediate |

The novel PanNETassigner NanoString assay achieved good-quality, reproducible results with a high level of concordance with subtyping results achieved using Microarray data.

The subtypes of 228-gene NanoString assay of PanNETassigner (NanoPanNETassigner; both by Pearson correlation and iLVM methods) assay were highly reproducible (0.96 Pearson correlation co-efficient). There was 95% concordance between NanoPanNETassigner and microarray subtypes.

### Example 2

### Survival Assessments in the Verona PanNET Cohort According to Subtype/Grade

Clinical data was available for 106 patients whose samples were assessed using the 228-gene NanoString assay. OS according to subtype and grade were assessed as outlined in FIGS. 2 and 3, and Table 9 below.

TABLE 9

| | | | | |
|---|---|---|---|---|
| | Median | 1 yr | 5 yr | 10 yr |
| No. | Survival | OS | OS | OS |
| Patients | Time | rate | rate | rate |

Subgroup

| | | | | | |
|---|---|---|---|---|---|
| Insulinoma | 37 | not reached | 100% | 95% | 95% |
| Intermediate | 40 | not reached | 98% | 89% | 89% |
| MLP | 29 | 71 months | 96% | 75% | 31% |

Grade

| | | | | | |
|---|---|---|---|---|---|
| 1 | 68 | not reached | 99% | 94% | 82% |
| 2 | 30 | not reached | 100% | 71% | 54% |
| 3 | 8 | 24 months | 86% | 0% | 0% |

The Kaplan-Meier Survival Curves were compared between subgroups and grades, as determined by Log Rank Hazard Ratio, are shown in Table 10.

TABLE 10

| | | | Log Rank | |
|---|---|---|---|---|
| | | | Hazard Ratio | P value |
| Subgroups Compared | | | | |
| | Insulinoma | Intermediate | 0.36 | 0.349 |
| | Insulinoma | MLP | 0.12 | 0.015 |
| | Intermediate | MLP | 0.035 | 0.114 |
| Grades Compared | | | | |
| | 1 | 2 | 0.48 | 0.296 |
| | 1 | 3 | 0.11 | <0.001 |
| | 2 | 3 | 0.08 | <0.001 |

The grade according to subtype in patients was then assessed using the 228-Gene NanoString Assay (n=106), and the results are shown in table 11.

TABLE 11

| | | | | |
|---|---|---|---|---|
| Subtype | Grade 1 | Grade 2 | Grade 3 | N |

| | | | | | | |
|---|---|---|---|---|---|---|
| Insulinoma | 28 (76%) | 6 | (16%) | 3 | (8%) | 37 |
| Intermediate | 28 (70%) | 11 | (27%) | 1 | (3%) | 40 |
| MLP | 12 (41%) | 13 | (45%) | 4 | (14%) | 29 |

Whilst 50% of the Grade 3 patients were MLPs, the MLP subtype also included Grade 1 and Grade 2 patients.

Discussion

Clinical data was available for 106 of the Verona Cohort tested using the 228-gene NanoString assay. Using Kaplan-Meier analysis the MLP patients had a significantly worse prognosis than the Insulinoma-like patients with a median OS of 71 months whereas OS was not reached for Insulinoma-like or Intermediate patients, which showed good prognosis.

Survival was also associated with Grade of disease with Grade 3 patients having a significantly worse median OS of 24 months, consistent with published data. It should be noted that only 14% of the MLP patients analysed had Grade 3 disease, demonstrating the ability of the PanNETassigner NanoString assay to highlight those patients with Grade 1 and 2 disease who have a worse prognosis than may be expected according to Grade alone.

Subtypes were independent predictor of OS, but with more grade-3 PanNETs in MLP.

Conclusion: NanoPanNETassigner assay defines robust and reproducible PanNETassigner subtypes with significant prognostic and mutational differences independent of grades. This assay with short turn-around time may facilitate prospective validation of subtypes in clinical trials.

### Example 3

### Determination of Gene Mutations Present in PanNET Subtypes

Using the NGS assay, recurrent gene alterations were found at different levels in the Insulinoma, Intermediate and MLP PanNET subtypes, and the results are shown in table 12.

TABLE 12

| ATM mutations | No. | Total | % |
|---|---|---|---|
| Insulinoma | 3 | 42 | 7% |
| Intermediate | 4 | 43 | 9% |
| MLP | 5 | 35 | 14% |
| DAXX/ATRX | No. | Total | % |
| Insulinoma | 3 | 42 | 7% |
| Intermediate | 15 | 43 | 35% |
| MLP | 7 | 35 | 20% |
| MEN1 | | Total | % |
| Insulinoma | 8 | 42 | 19% |
| Intermediate | 23 | 43 | 53% |
| MLP | 9 | 35 | 26% |
| mTOR pathway | | | |
| (TSC1/TSC2/PTEN) | | Total | % |
| Insulinoma | 1 | 42 | 2% |
| Intermediate | 9 | 43 | 21% |
| MLP | 9 | 35 | 26% |

MEN1 mutations are significantly enriched in the intermediate subtype. DAXX/ATRX mutations significantly associated with MLP and intermediate subtype. TSC2/PTEN/ATM mutations are associated with MLP and intermediate subtypes.

### Example 4

### Reduction of Gene Sets as Biomarkers

In an effort to identify a robust smaller set of genes for assigning samples into PanNETassigner subtypes, we selected a robust set of samples using Silhouette statistical method⁴⁰.

Next, we selected a robust set of genes that best predict the PanNETassigner subtypes with lowest misclassification error rate (MCR) using the robust samples selected from Silhouette and another in-house built R package, intPredict. intPredict employed a pipeline of different gene selection and class prediction methods to develop a robust gene classifier to predict subtypes by randomly splitting the original data set of samples into training and test data sets and executing the pipeline repeatedly 50 or more times. Gene selection methods included prediction strength (PS)⁴¹, Prediction Analysis of Microarrays PAM⁴² and between-within group sum of squares ratio (BW)^{→}. Furthermore, the best performing gene set from the gene selection methods was identified using multiple class prediction methods such as random forest (RF)⁴⁴, diagonal linear discriminant analysis (DLDA)⁴³ and two support vector machines (SVM) approaches—linear and radial methods⁴⁵. The gene set with the lowest MCR was determined as follows,

$$MCR = \frac{1}{k} {\sum }_{i = 1}^{k} {e}_{i} \qquad ( 1 )$$

where k is the number of test samples, and eᵢ is the misclassification of each test sample compared to known subtype.

R package e1071 (v1.6-8)⁴⁶ was utilised for both SVM methods; randomForest (v4.6-12)⁴⁷ for RF; sma (v0.5.17)⁴⁸ for BW and DLDA; and pamr (v1.55)⁴⁹ for PAM. An R package idSample is available at github https://github.com/syspremed/idSample, and intPredict at https://github.com/syspremed/intPredict.

The results are shown in table 13.

TABLE 13
Misclassification error rates

| Number of | Method within | Misclassification | |
|---|---|---|---|
| genes | intPredict | Error Rate | Genes |
| 2 | BW-SVMrd | 0.24 | GLS, GRM5 |
| 4 | PS-SVMrd | 0.15 | GLS, GRM5, MOBKL1A, USP29 |
| 6 | PS-SVMrd | 0.14 | CTRL, GLS, GRM5, MASP2, MOBKL1A, |
| | | | USP29 |
| 8 | pam-SVMln | 0.12 | CPA1, CTRL, GLS, GRM5, MASP2, |
| | | | MOBKL1A, PNLIPRP1, USP29 |
| 10 | BW-SVMrd | 0.12 | CPA1, CPA2, CTRC, CTRL, GLS, GRM5, |
| | | | MASP2, MOBKL1A, PNLIPRP1, USP29 |
| 15 | BW-SVMrd | 0.1 | CPA1, CPA2, CTRL, CYP4F3, GLS, GRM5, |
| | | | HAO1, KLK4, MAFB, MASP2, MOBKL1A, |
| | | | PNLIPRP1, SERPIND1, STEAP3, USP29 |
| 20 | pam-SVMln | 0.08 | ANGPTL3, APOH, CLDN10, CLPS, CPA1, |
| | | | CPA2, CTRC, CTRL, CYP4F3, GP2, GRM5, |
| | | | HAO1, INS, MASP2, PDIA2, PNLIP, |
| | | | PNLIPRP1, PNLIPRP2, SERPIND1, USP29 |
| 30 | pam-SVMln | 0.04 | ANGPTL3, APOH, CLDN10, CLPS, CPA1, |
| | | | CPA2, CRYBA2, CTRC, CTRL, CYP4F3, |
| | | | EGLN3, GLP1R, GP2, GRM5, HAO1, INS, |
| | | | LOXL4, MASP2, P2RX1, PDIA2, PNLIP, |
| | | | PNLIPRP1, PNLIPRP2, REG1B, SERPINA1, |
| | | | SERPIND1, SERPINI2, STEAP3, TFF1, |
| | | | USP29, |
| 40 | pam-SVMln | 0.02 | ANGPTL3, APOH, C19orf77, CELA1, |
| | | | CLDN10, CLPS, CPA1, CPA2, CRYBA2, |
| | | | CTRC, CTRL, CYP4F3, EGLN3, ENTPD3, |
| | | | GCGR, GLP1R, GLS, GP2, GRM5, HAO1, |
| | | | INS, KLK4, LOXL4, MAFB, MASP2, MIA2, |
| | | | P2RX1, PDIA2, PNLIP, PNLIPRP1, |
| | | | PNLIPRP2, REG1B, SCD5, SERPINA1, |
| | | | SERPIND1, SERPINI2, STEAP3, TFF1, |
| | | | TMPRSS4, USP29 |
| 50 | pam-SVMln | 0.01 | ALDH1A1, ANGPTL3, APOH, C19orf77, |
| | | | CEACAM1, CELA1, CLDN10, CLPS, CPA1, |
| | | | CPA2, CRYBA2, CTRC, CTRL, CYP4F3, |
| | | | DAPL1, EGLN3, ELSPBP1, ENTPD3, GCGR, |
| | | | GLP1R, GLS, GP2, GRM5, HAO1, INS, |
| | | | KLK4, LOXL4, MAFB, MASP2, MIA2, |
| | | | MOBKL1A, P2RX1, PDIA2, PNLIP, |
| | | | PNLIPRP1, PNLIPRP2, PRLR, RBP4, |
| | | | REG1B, SCD5, SERPINA1, SERPIND1, |
| | | | SERPINI2, SLC16A3, STEAP3, TFF1, |
| | | | TM4SF4, TM4SF5, TMPRSS4, USP29 |
| 100 | BW-SVMln | 0.01 | A1CF, ACVR1B, ADAM28, ADM, ALDH1A1, |
| | | | ANGPTL3, APOH, ARRDC4, BTC, C19orf77, |
| | | | C20orf46, CEACAM1, CELA1, CHST1, |
| | | | CLDN10, CLPS, COL8A1, CPA1, CPA2, |
| | | | CRYBA2, CTRC, CTRL, CYP4F3, DAPL1, |
| | | | EGFR, EGLN3, ELSPBP1, ENTPD3, ERBB3, |
| | | | F10, F7, FKBP11, GATM, GCGR, GLP1R, |
| | | | GLS, GP2, GRM5, HAO1, HSD11B2, |
| | | | IL20RA, INS, KLK4, LOXL4, LRAT, MAFB, |
| | | | MASP2, MIA2, MNX1, MOBKL1A, MX2, |
| | | | NUPR1, P2RX1, PDGFC, PDIA2, PEMT, |
| | | | PFKFB2, PFKFB3, PLIN3, PMEPA1, PNLIP, |
| | | | PNLIPRP1, PNLIPRP2, PRLR, RARRES2, |
| | | | RBP4, REG1B, ROBO3, SCD5, SERPINA1, |
| | | | SERPINA3, SERPIND1, SERPINI2, SH3BP4, |
| | | | SLC16A3, SLC2A1, SLC30A2, SLC7A2, |
| | | | SLC7A8, SMARCA1, SMOC2, SSX2IP, |
| | | | STEAP3, SUSD5, TACSTD2, TBC1D24, |
| | | | TFF1, TGFBR3, TGIF1, TM4SF1, TM4SF4, |
| | | | TM4SF5, TMEM176B, TMEM181, TMEM90B, |
| | | | TMPRSS4, TSHZ3, USP29, VEGFC, WNT4 |

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. Young K, Iyer R, Morganstein D, Chau I, Cunningham O, Starling N. Pancreatic neuroendocrine tumors: a review. Futur Oncol. 2015; 11(5):853-864. doi:10.2217/fon.14.285.

2. Modlin I M, Lye K D, Kidd M. A 5-decade analysis of 13,715 carcinoid tumors. Cancer. 2003; 97(4):934-959. doi:10.1002/cncr.11105.

3. Yao J C, Hassan M, Phan A, et al. One hundred years after “carcinoid”: epidemiology of and prognostic factors for neuroendocrine tumors in 35,825 cases in the United States. J Clin Oncol. 2008; 26(18):3063-3072. doi:10.1200/JCO.2007.15.4377.

4. Ekeblad S, Skogseid B, Dunder K, Oberg K, Eriksson B. Prognostic Factors and Survival in 324 Patients with Pancreatic Endocrine Tumor Treated at a Single Institution. Clin Cancer Res. 2008; 14(23):7798-7803. doi:10.1158/1078-0432.CCR-08-0734.

5. Oberg K, Knigge U, Kwekkeboom D, Perren A, ESMO Guidelines Working Group. Neuroendocrine gastro-entero-pancreatic tumors: ESMO Clinical Practice Guidelines for diagnosis, treatment and follow-up. Ann Oncol. 2012; 23(suppl 7):vii124-vii130. doi:10.1093/annonc/mds295.

6. Scarpa A, Mantovani W, Capelli P, et al. Pancreatic endocrine tumors: improved TNM staging and histopathological grading permit a clinically efficient prognostic stratification of patients. Mod Pathol. 2010; 23(6):824-833. doi:10.1038/modpathol.2010.58.

7. Pavel M, O'Toole D, Costa F, et al. ENETS Consensus Guidelines Update for the Management of Distant Metastatic Disease of Intestinal, Pancreatic, Bronchial Neuroendocrine Neoplasms (NEN) and NEN of Unknown Primary Site. Neuroendocrinology. 2016; 103(2):172-185. doi:10.1159/000443167.

8. Delle Fave G, O'Toole D, Sundin A, et al. ENETS Consensus Guidelines Update for Gastroduodenal Neuroendocrine Neoplasms. Neuroendocrinology. 2016; 103(2):119-124. doi:10.1159/000443168.

9. Niederle B, Pape U-F, Costa F, et al. ENETS Consensus Guidelines Update for Neuroendocrine Neoplasms of the Jejunum and Ileum. Neuroendocrinology. 2016; 103(2):125-138. doi:10.1159/000443170.

10. Falconi M, Eriksson B, Kaltsas G, et al. ENETS Consensus Guidelines Update for the Management of Patients with Functional Pancreatic Neuroendocrine Tumors and Non-Functional Pancreatic Neuroendocrine Tumors. Neuroendocrinology. 2016; 103(2):153-171. doi:10.1159/000443171.

11. Ricci C, Casadei R, Taffurelli G, et al. WHO 2010 classification of pancreatic endocrine tumors. is the new always better than the old? Pancreatology. 14(6):539-541. doi:10.1016/j.pan.2014.09.005.

12. Hauck L, Bitzer M, Malek N, Plentz R R. Subgroup analysis of patients with G2 gastroenteropancreatic neuroendocrine tumors. Scand J Gastroenterol. July 2015:1-5. doi:10.3109/00365521.2015.1064994.

13. Reid M D, Balci S, Saka B, Adsay N V. Neuroendocrine tumors of the pancreas: current concepts and controversies. Endocr Pathol. 2014; 25(1):65-79. doi:10.1007/s12022-013-9295-2.

14. Young et al. A Single Institution Experience of Treating Gastroenteropancreatic Neuroendocrine Tumors (GEP-NETs) over the Last 10 Years: The Royal Marsden Hospital (RM) Experience.

15. International Agency for Research on Cancer, Lloyd R V., Osamura R Y, Kloppel G. Who Classification of Tumors of Endocrine Organs. World Health Organization; 2017. http://publications.iarc.fr/Book-And-Report-Series/Who-Iarc-Classification-Of-Tumors/Who-Classification-Of-Tumors-Of-Endocrine-Organs-2017. Accessed Oct. 23, 2017.

16. Scarpa A, Chang D K, Nones K, et al. Whole-genome landscape of pancreatic neuroendocrine tumors. Nature. 2017; 543(7643):65-71. doi:10.1038/nature21063.

17. Jiao Y, Shi C, Edil B H, et al. DAXX/ATRX, MEN1, and mTOR pathway genes are frequently altered in pancreatic neuroendocrine tumors. Science. 2011; 331(6021):1199-1203. doi:10.1126/science.1200609.

18. Chan D L, Clarke S J, Diakos C I, et al. Prognostic and predictive biomarkers in neuroendocrine tumors. Crit Rev Oncol. 2017; 113:268-282. doi:10.1016/j.critrevonc.2017.03.017.

19. Marinoni I, Kurrer A S, Vassella E, et al. Loss of DAXX and ATRX are associated with chromosome instability and reduced survival of patients with pancreatic neuroendocrine tumors. Gastroenterology. 2014; 146(2):453-60.e5. doi:10.1053/j.gastro.2013.10.020.

20. Singhi A D, Liu T-C, Roncaioli J L, et al. Alternative Lengthening of Telomeres and Loss of DAXX/ATRX Expression Predicts Metastatic Disease and Poor Survival in Patients with Pancreatic Neuroendocrine Tumors. Clin Cancer Res. 2017; 23(2):600-609. doi:10.1158/1078-0432.CCR-16-1113.

21. Park J K, Paik W H, Lee K, Ryu J K, Lee S H, Kim Y-T. DAXX/ATRX and MEN1 genes are strong prognostic markers in pancreatic neuroendocrine tumors. Oncotarget. 2017; 8(30):49796-49806. doi:10.18632/oncotarget.17964.

22. Sadanandam A, Wullschleger S, Lyssiotis C A, et al. A Cross-Species Analysis in Pancreatic Neuroendocrine Tumors Reveals Molecular Subtypes with Distinctive Clinical, Metastatic, Developmental, and Metabolic Characteristics. Cancer Discov. 2015; 5(12):1296-1313. doi:10.1158/2159-8290.CD-15-0068.

40. Rousseeuw P J. Silhouettes: A graphical aid to the interpretation and validation of cluster analysis. Journal of Computational and Applied Mathematics 1987; 20: 53-65.

41. Golub T R, Slonim D K, Tamayo P, Huard C, Gaasenbeek M, Mesirov J P, Coller H, Loh M L, Downing J R, Caligiuri M A. Molecular classification of cancer: class discovery and class prediction by gene expression monitoring. Science 1999; 286: 531-7.

42. Tibshirani R, Hastie T, Narasimhan B, Chu G. Diagnosis of multiple cancer types by shrunken centroids of gene expression. Proceedings of the National Academy of Sciences 2002; 99: 6567-72.

43. Dudoit S, Fridlyand J, Speed T P. Comparison of discrimination methods for the classification of tumors using gene expression data. Journal of the American Statistical Association 2002; 97: 77-87.

44. Breiman L. Random forests. Machine Learning 2001; 45: 5-32.

45. Cortes C, Vapnik V. Support-vector networks. Machine Learning 1995; 20: 273-97.

46. Meyer D, Dimitriadou E, Hornik K, Weingessel A, Leisch F, Chang C-C, Lin C-C, Meyer M D. Package ‘e1071’, 2017.

47. Liaw A, Wiener M. Classification and regression by randomForest. R news 2002; 2: 18-22.

48. Dudoit S, Yang Y, Bolstad B. sma: Statistical microarray analysis. https://cranr-projectorg/package=sma 2011.

49. Nielsen T, Wallden B, Schaper C, Ferree S, Liu S, Gao D, Barry G, Dowidar N, Maysuria M, Storhoff J, Henry N, Hayes D, et al. Analytical validation of the PAM50-based Prosigna Breast Cancer Prognostic Gene Signature Assay and nCounter Analysis System using formalin-fixed paraffin-embedded breast tumor specimens. BMC Cancer 2014; 14: 177.

1. A method for predicting the prognosis of a human pancreatic neuroendocrine tumor (PanNET) patient, the method comprising:
a) measuring the gene expression of at least 30 genes selected from: GLS, GRM5, CEACAM1, INS, PFKFB2, ELSPBP1, MIA2, ENTPD3, STEAP3, APOH, SERPINA1, A1CF, PRLR, F10, TMEM176B, MASP2, RBP4, CYP4F3, CHST8, KLK4, USP29, CELA1, TM4SF4, TMPRSS4, SCD5, TM4SF5, SERPIND1, P2RX1, GLP1R, LRAT, CASR, DAPL1, ERBB3, C19orf77, F7, PLIN3, NEFM, MNX1, ROBO3, CPA1, CTRL, TGFBR3, PNLIPRP2, TSHZ3, ADAMTS2, GLRA2, HGD, GP2, CTRC, RAB17, ANGPTL3, LOXL4, PNLIP, PEMT, CPA2, PNLIPRP1, ALDH1A1, SLC12A7, IL20RA, CLPS, C20orf46, GCGR, IL18R1, PDIA2, NAAA, BTC, TAPBPL, ELMO1, KLK8, CDS1, TFF1, TBC1D24, KIT, MOBKL1A, PLA1A, SUSD5, CRYBA2, PMM1, EFNA1, SLC16A3, FKBP11, IL22RA1, ADM, EGLN3, LGALS4, TLE2, CLDN10, NUPR1, SERPINI2, PTPLA, PVRL4, EGFR, MAFB, PFKFB3, HSD11B2, FGB, NDC80, SMOC2, ACVR1B, TGIF1, ARRDC4, MMP1, TACSTD2, TOP2A, SH3BP4, PDGFC, THBS2, CNPY2, HAO1, ADAM28, C7orf68, GATM, CXCR4, PAFAH1B3, NEK6, AKR1C4, F12, PMEPA1, RAB7L1, SMO, CLDN1, CHST1, WNT4, TMPRSS15, SPAG4, MX2, SLC7A2, GUCA1C, SLC7A8, PRSS22, RARRES2, PRSS8, SLC30A2, TMEM90B, VIPR2, CXCR7, SMARCA1, FAM19A5, CLDN11, SERPINA3, GAL3ST4, AFG3L1, COL8A1, SSX2IP, IMPA2, VEGFC, TMEM181, LGALS2, PLXDC1, TLR3, PSMB9, CHI3L2, PLCE1, ABI3BP, NUDT5, FOXO4, SLC2A1, COL1A2, REG1B, NETO2, ENC1, DLL1, TM4SF1, CKS2, FGD1, PPEF1, LEF1, MLN, TNFAIP6, ACAD9, TYMS, ZNF521, ACADSB, TSC2, HR, DEFB1, GRSF1, ACE, SRGAP3, SMEK1, TWIST1, FMNL1, ADAMTS7, COL5A2, IFI44, CAPN13, AQP8, IP6K2, COPE, MXRA5, RBPJL, MBP, MAP3K14, CLCA1, IDS, TECR, CAPNS1 and POSTN, in a sample obtained from the PanNET of the patient to obtain a sample gene expression profile of at least said genes; and
b) making a prediction of the prognosis of the patient based on the sample gene expression profile.

2. A method according to claim 1, wherein the at least 30 genes include any or all of:
(a) A1CF, ACVR1B, ADAM28, ADM, ALDH1A1, ANGPTL3, APOH, ARRDC4, BTC, C19orf77, C20orf46, CEACAM1, CELA1, CHST1, CLDN10, CLPS, COL8A1, CPA1, CPA2, CRYBA2, CTRC, CTRL, CYP4F3, DAPL1, EGFR, EGLN3, ELSPBP1, ENTPD3, ERBB3, F10, F7, FKBP11, GATM, GCGR, GLP1R, GLS, GP2, GRM5, HAO1, HSD11B2, IL20RA, INS, KLK4, LOXL4, LRAT, MAFB, MASP2, MIA2, MNX1, MOBKL1A, MX2, NUPR1, P2RX1, PDGFC, PDIA2, PEMT, PFKFB2, PFKFB3, PLIN3, PMEPA1, PNLIP, PNLIPRP1, PNLIPRP2, PRLR, RARRES2, RBP4, REG1B, ROBO3, SCD5, SERPINA1, SERPINA3, SERPIND1, SERPINI2, SH3BP4, SLC16A3, SLC2A1, SLC30A2, SLC7A2, SLC7A8, SMARCA1, SMOC2, SSX2IP, STEAP3, SUSD5, TACSTD2, TBC1D24, TFF1, TGFBR3, TGIF1, TM4SF1, TM4SF4, TM4SF5, TMEM176B, TMEM181, TMEM90B, TMPRSS4, TSHZ3, USP29, VEGFC, WNT4;
(b) ALDH1A1, ANGPTL3, APOH, C19orf77, CEACAM1, CELA1, CLDN10, CLPS, CPA1, CPA2, CRYBA2, CTRC, CTRL, CYP4F3, DAPL1, EGLN3, ELSPBP1, ENTPD3, GCGR, GLP1R, GLS, GP2, GRM5, HAO1, INS, KLK4, LOXL4, MAFB, MASP2, MIA2, MOBKL1A, P2RX1, PDIA2, PNLIP, PNLIPRP1, PNLIPRP2, PRLR, RBP4, REG1B, SCD5, SERPINA1, SERPIND1, SERPINI2, SLC16A3, STEAP3, TFF1, TM4SF4, TM4SF5, TMPRSS4, USP29;
(c) ANGPTL3, APOH, C19orf77, CELA1, CLDN10, CLPS, CPA1, CPA2, CRYBA2, CTRC, CTRL, CYP4F3, EGLN3, ENTPD3, GCGR, GLP1R, GLS, GP2, GRM5, HAO1, INS, KLK4, LOXL4, MAFB, MASP2, MIA2, P2RX1, PDIA2, PNLIP, PNLIPRP1, PNLIPRP2, REG1B, SCD5, SERPINA1, SERPIND1, SERPINI2, STEAP3, TFF1, TMPRSS4, USP29;
(d) ANGPTL3, APOH, CLDN10, CLPS, CPA1, CPA2, CRYBA2, CTRC, CTRL, CYP4F3, EGLN3, GLP1R, GP2, GRM5, HAO1, INS, LOXL4, MASP2, P2RX1, PDIA2, PNLIP, PNLIPRP1, PNLIPRP2, REG1B, SERPINA1, SERPIND1, SERPINI2, STEAP3, TFF1, USP29
(e) ANGPTL3, APOH, CLDN10, CLPS, CPA1, CPA2, CTRC, CTRL, CYP4F3, GP2, GRM5, HAO1, INS, MASP2, PDIA2, PNLIP, PNLIPRP1, PNLIPRP2, SERPIND1, USP29,
(f) CPA1, CPA2, CTRL, CYP4F3, GLS, GRM5, HAO1, KLK4, MAFB, MASP2, MOBKL1A, PNLIPRP1, SERPIND1, STEAP3, USP29;
(g) CPA1, CPA2, CTRC, CTRL, GLS, GRM5, MASP2, MOBKL1A, PNLIPRP1, USP29;
(h) CPA1, CTRL, GLS, GRM5, MASP2, MOBKL1A, PNLIPRP1, USP29;
(i) CTRL, GLS, GRM5, MASP2, MOBKL1A, USP29;
(j) GLS, GRM5, MOBKL1A, USP29; or
(k) GLS, GRM5.

3. The method of claim 1, wherein step b) making a prediction of the prognosis of the patient based on the sample gene expression profile comprises:
(i) optionally, normalising the measured expression level of each gene relative to the expression level of one or more housekeeping genes;
(ii) comparing the sample gene expression profile, optionally after said normalising, with one or more reference centroids comprising:
a first reference centroid that represents the summarised gene expression of the measured genes in an ‘insulinoma-like’ type patient;
a second reference centroid that represents the summarised gene expression of the measured genes in an ‘intermediate’ type patient;
a third reference centroid that represents the summarised gene expression of the measured genes in a ‘metastasis-like-primary’ (MLP) type patient;
iii) classifying the sample gene expression profile as belonging to the insulinoma-like, intermediate or MLP group having the reference centroid to which it is most closely matched; and
iv) providing a prognosis based on the classification made in step iii).

4. The method of claim 2, wherein the reference centroids have been pre-determined and are obtained by retrieval from a volatile or non-volatile computer memory or data store.

5. The method of claim 3, wherein the reference centroids comprise one, two or all three of the following centroids:

| genes | Insulinoma-like | Intermediate | MLP |
|---|---|---|---|
| CEACAM1 | −2.619 | 0.5175 | 0.4646 |
| INS | 2.1656 | −0.5311 | −0.281 |
| PFKFB2 | 2.0939 | −0.481 | −0.3042 |
| ELSPBP1 | 2.087 | −0.3975 | −0.3851 |
| MIA2 | −2.0783 | 0.6246 | 0.1547 |
| ENTPD3 | 2.0695 | −0.3349 | −0.4412 |
| GRM5 | 1.9661 | −0.4081 | −0.3292 |
| STEAP3 | 1.8861 | −0.6741 | −0.0332 |
| APOH | −1.843 | 0.7066 | −0.0155 |
| SERPINA1 | −1.8421 | 0.6017 | 0.0891 |
| A1CF | −1.8091 | 0.4938 | 0.1846 |
| PRLR | −1.7938 | 0.4453 | 0.2274 |
| F10 | −1.7023 | 0.6704 | −0.032 |
| TMEM176B | −1.6658 | 0.3388 | 0.2859 |
| MASP2 | 1.6557 | −0.4494 | −0.1715 |
| RBP4 | 1.5705 | −0.7774 | 0.1884 |
| CYP4F3 | −1.543 | 0.4915 | 0.0871 |
| CHST8 | 1.5392 | −0.2847 | −0.2925 |
| KLK4 | 1.5317 | −0.4333 | −0.1411 |
| USP29 | 1.5013 | −0.3892 | −0.1737 |
| CELA1 | 1.4676 | −0.5537 | 0.0033 |
| TM4SF4 | −1.4098 | 0.2599 | 0.2687 |
| TMPRSS4 | 1.3881 | −0.4395 | −0.0811 |
| SCD5 | 1.3817 | −0.3667 | −0.1515 |
| TM4SF5 | −1.3527 | 0.151 | 0.3563 |
| SERPIND1 | −1.2469 | 0.5658 | −0.0982 |
| P2RX1 | 1.2378 | −0.567 | 0.1028 |
| GLP1R | 1.227 | −0.7076 | 0.2475 |
| LRAT | −1.2001 | 0.3925 | 0.0576 |
| CASR | 1.1903 | −0.4101 | −0.0363 |
| DAPL1 | 1.1772 | −0.394 | −0.0474 |
| ERBB3 | −1.1551 | 0.2507 | 0.1824 |
| C19orf77 | −1.1366 | 0.5365 | −0.1103 |
| F7 | −1.1088 | 0.4146 | 0.0012 |
| PLIN3 | −1.1061 | 0.3651 | 0.0496 |
| NEFM | 1.0914 | −0.4468 | 0.0375 |
| MNX1 | 1.0502 | −0.187 | −0.2068 |
| ROBO3 | 1.0498 | −0.4796 | 0.0859 |
| CPA1 | 1.0396 | −0.171 | −0.2189 |
| CTRL | 1.0324 | −0.2598 | −0.1274 |
| TGFBR3 | 1.0314 | −0.3271 | −0.0597 |
| PNLIPRP2 | 1.0293 | −0.3144 | −0.0716 |
| TSHZ3 | 0.9894 | −0.5562 | 0.1852 |
| ADAMTS2 | 0.9775 | −0.1468 | −0.2198 |
| GLRA2 | −0.9719 | 0.444 | −0.0796 |
| HGD | −0.9546 | 0.1951 | 0.1629 |
| GP2 | 0.9486 | −0.1884 | −0.1674 |
| CTRC | 0.9472 | −0.1359 | −0.2193 |
| RAB17 | −0.943 | 0.1644 | 0.1892 |
| ANGPTL3 | −0.9309 | 0.7313 | −0.3822 |
| LOXL4 | −0.9227 | 0.8894 | −0.5434 |
| PNLIP | 0.9217 | −0.1173 | −0.2283 |
| PEMT | −0.9181 | 0.1348 | 0.2094 |
| CPA2 | 0.898 | −0.1357 | −0.201 |
| PNLIPRP1 | 0.89 | −0.2451 | −0.0887 |
| ALDH1A1 | −0.888 | 0.4516 | −0.1186 |
| SLC12A7 | −0.8633 | 0.048 | 0.2757 |
| IL20RA | 0.8596 | −0.6899 | 0.3675 |
| CLPS | 0.8537 | −0.0882 | −0.232 |
| GLS | −0.8338 | 0.6425 | −0.3299 |
| C20orf46 | −0.8229 | 0.0879 | 0.2207 |
| GCGR | 0.8167 | −0.3211 | 0.0149 |
| IL18R1 | −0.8071 | 0.3806 | −0.078 |
| PDIA2 | 0.8067 | −0.2371 | −0.0655 |
| NAAA | −0.801 | 0.0699 | 0.2304 |
| BTC | −0.777 | 0.3415 | −0.0501 |
| TAPBPL | −0.7718 | 0.1346 | 0.1548 |
| ELMO1 | 0.7599 | −0.1868 | −0.0982 |
| KLK8 | −0.7466 | 0.3572 | −0.0772 |
| CDS1 | −0.7344 | 0.1808 | 0.0946 |
| TFF1 | −0.4502 | −0.5565 | 0.7253 |
| TBC1D24 | 0.7087 | −0.2012 | −0.0646 |
| KIT | −0.1886 | −0.6275 | 0.6983 |
| MOBKL1A | −0.6906 | 0.5167 | −0.2577 |
| PLA1A | −0.6807 | 0.0925 | 0.1627 |
| SUSD5 | 0.6571 | −0.4075 | 0.1611 |
| CRYBA2 | 0.0085 | 0.6535 | −0.6567 |
| PMM1 | −0.6512 | 0.129 | 0.1152 |
| EFNA1 | −0.6482 | −0.0629 | 0.3059 |
| SLC16A3 | −0.3093 | −0.5288 | 0.6448 |
| FKBP11 | −0.6405 | 0.2467 | −0.0065 |
| IL22RA1 | 0.0157 | −0.6362 | 0.6303 |
| ADM | −0.4275 | −0.4641 | 0.6244 |
| EGLN3 | −0.622 | −0.3749 | 0.6082 |
| LGALS4 | 0.2964 | −0.6215 | 0.5104 |
| TLE2 | −0.6031 | 0.2808 | −0.0546 |
| CLDN10 | 0.6022 | −0.2928 | 0.067 |
| NUPR1 | −0.0905 | −0.5664 | 0.6003 |
| SERPINI2 | 0.599 | −0.2985 | 0.0739 |
| PTPLA | −0.5914 | 0.1826 | 0.0392 |
| PVRL4 | 0.5913 | −0.4074 | 0.1857 |
| EGFR | −0.5301 | −0.3817 | 0.5805 |
| MAFB | 0.5783 | 0.2629 | −0.4798 |
| PFKFB3 | −0.2536 | −0.4824 | 0.5775 |
| HSD11B2 | 0.4836 | −0.5774 | 0.396 |
| FGB | −0.5585 | 0.1894 | 0.02 |
| NDC80 | −0.5544 | −0.3437 | 0.5517 |
| SMOC2 | 0.0794 | −0.5528 | 0.523 |
| ACVR1B | 0.4536 | −0.5522 | 0.3821 |
| TGIF1 | 0.2595 | −0.5502 | 0.4529 |
| ARRDC4 | −0.5175 | 0.4019 | −0.2078 |
| MMP1 | 0.2828 | −0.5127 | 0.4066 |
| TACSTD2 | 0.5006 | −0.4165 | 0.2288 |
| TOP2A | 0.2935 | −0.492 | 0.3819 |
| SH3BP4 | −0.0613 | −0.4678 | 0.4908 |
| PDGFC | 0.1177 | −0.4879 | 0.4437 |
| THBS2 | −0.2884 | −0.3781 | 0.4863 |
| CNPY2 | −0.4827 | 0.0704 | 0.1106 |
| HAO1 | −0.1631 | 0.4717 | −0.4105 |
| ADAM28 | 0.0504 | −0.4669 | 0.448 |
| C7orf68 | −0.4065 | −0.312 | 0.4644 |
| GATM | 0.4616 | −0.3139 | 0.1408 |
| CXCR4 | −0.1765 | −0.3947 | 0.4609 |
| PAFAH1B3 | −0.4603 | 0.0567 | 0.1159 |
| NEK6 | −0.4529 | −0.2507 | 0.4205 |
| AKR1C4 | −0.2208 | −0.3692 | 0.452 |
| F12 | −0.4515 | −0.1248 | 0.2941 |
| PMEPA1 | 0.449 | −0.4494 | 0.281 |
| RAB7L1 | 0.4491 | 0.0954 | −0.2638 |
| SMO | −0.0939 | −0.4117 | 0.4469 |
| CLDN1 | −0.4422 | 0.0249 | 0.1409 |
| CHST1 | 0.4421 | −0.3476 | 0.1818 |
| WNT4 | −0.231 | 0.4383 | −0.3517 |
| TMPRSS15 | −0.2167 | −0.3553 | 0.4365 |
| SPAG4 | −0.4348 | −0.1291 | 0.2921 |
| MX2 | −0.0034 | −0.4324 | 0.4337 |
| SLC7A2 | −0.076 | 0.4293 | −0.4008 |
| GUCA1C | −0.4275 | 0.2248 | −0.0645 |
| SLC7A8 | 0.4251 | 0.1764 | −0.3358 |
| PRSS22 | 0.4232 | −0.2329 | 0.0742 |
| RARRES2 | 0.1893 | −0.42 | 0.349 |
| PRSS8 | −0.4163 | 0.1247 | 0.0315 |
| SLC30A2 | 0.2978 | −0.4142 | 0.3025 |
| TMEM90B | −0.0705 | 0.4091 | −0.3827 |
| VIPR2 | 0.2079 | −0.4031 | 0.3251 |
| CXCR7 | −0.0836 | −0.3682 | 0.3996 |
| SMARCA1 | −0.3969 | 0.3089 | −0.1601 |
| FAM19A5 | −0.0086 | −0.3846 | 0.3878 |
| CLDN11 | 0.3874 | −0.0013 | −0.144 |
| SERPINA3 | 0.2386 | −0.3838 | 0.2944 |
| GAL3ST4 | −0.3788 | 0.0897 | 0.0523 |
| AFG3L1 | −0.376 | 0.1502 | −0.0092 |
| COL8A1 | −0.0067 | −0.3662 | 0.3687 |
| SSX2IP | −0.3254 | 0.368 | −0.2459 |
| IMPA2 | −0.2547 | −0.2701 | 0.3656 |
| VEGFC | −0.2604 | 0.3522 | −0.2546 |
| TMEM181 | 0.3434 | −0.2532 | 0.1245 |
| LGALS2 | 0.2734 | −0.3411 | 0.2386 |
| PLXDC1 | −0.1591 | −0.2811 | 0.3408 |
| TLR3 | 0.0666 | −0.3357 | 0.3108 |
| PSMB9 | −0.2906 | −0.2264 | 0.3354 |
| CHI3L2 | 0.3323 | −0.2335 | 0.1089 |
| PLCE1 | 0.3321 | −0.0457 | −0.0788 |
| ABI3BP | −0.3227 | 0.0663 | 0.0547 |
| NUDT5 | 0.3208 | −0.0512 | −0.0691 |
| FOXO4 | −0.3167 | −0.146 | 0.2647 |
| SLC2A1 | −0.149 | −0.2605 | 0.3164 |
| COL1A2 | 0.052 | −0.3153 | 0.2958 |
| REG1B | 0.3082 | −0.1317 | 0.0162 |
| NETO2 | −0.2815 | −0.2013 | 0.3069 |
| ENC1 | −0.1294 | −0.2538 | 0.3023 |
| DLL1 | −0.2356 | −0.1945 | 0.2829 |
| TM4SF1 | 0.0249 | −0.2812 | 0.2718 |
| CKS2 | 0.0047 | −0.2754 | 0.2737 |
| FGD1 | −0.2749 | −0.0247 | 0.1278 |
| PPEF1 | −0.2541 | −0.1781 | 0.2734 |
| LEF1 | −0.1015 | −0.2324 | 0.2704 |
| MLN | 0.1306 | −0.2663 | 0.2173 |
| TNFAIP6 | −0.2658 | −0.1274 | 0.2271 |
| ACAD9 | 0.2533 | −0.1142 | 0.0192 |
| TYMS | −0.2394 | −0.1627 | 0.2525 |
| ZNF521 | −0.2491 | 0.0771 | 0.0163 |
| ACADSB | 0.2474 | −0.1114 | 0.0187 |
| TSC2 | 0.2426 | 0.0098 | −0.1008 |
| HR | 0.0515 | −0.2371 | 0.2178 |
| DEFB1 | −0.0916 | −0.1918 | 0.2262 |
| GRSF1 | −0.1592 | 0.2219 | −0.1622 |
| ACE | −0.2182 | 0.0208 | 0.061 |
| SRGAP3 | 0.2144 | −0.072 | −0.0084 |
| SMEK1 | −0.2144 | 0.0146 | 0.0658 |
| TWIST1 | −0.0591 | −0.1706 | 0.1928 |
| FMNL1 | 0.1916 | −0.1785 | 0.1067 |
| ADAMTS7 | −0.1902 | 0.0895 | −0.0182 |
| COL5A2 | 0.118 | −0.1878 | 0.1435 |
| IFI44 | −0.175 | −0.0689 | 0.1345 |
| CAPN13 | 0.0494 | −0.1671 | 0.1486 |
| AQP8 | 0.1354 | 0.1002 | −0.151 |
| IP6K2 | 0.1456 | −0.0236 | −0.031 |
| COPE | −0.1402 | 0.0235 | 0.0291 |
| MXRA5 | −0.1284 | −0.0335 | 0.0817 |
| RBPJL | 0.019 | 0.1183 | −0.1255 |
| MBP | −0.0392 | −0.1016 | 0.1163 |
| MAP3K14 | 0.0979 | −0.1025 | 0.0658 |
| CLCA1 | 0.0703 | −0.0936 | 0.0672 |
| IDS | 0.0688 | 0.0215 | −0.0473 |
| TECR | 0.0606 | 0.0193 | −0.042 |
| CAPNS1 | −0.0055 | −0.0539 | 0.0559 |
| POSTN | −0.0558 | 0.0271 | −0.0062 |

6. The method of claim 3, wherein when the sample gene expression profile is classified as MLP the patient is at high risk of metastasis.

7. The method of claim 3, wherein when the sample gene expression profile is classified as:
(i) insulinoma-like, the patient is at low risk of poor prognosis;
(ii) intermediate, the patient is at low risk of a poor prognosis; and
(iii) MLP, the patient is at high risk of poor prognosis.

8. The method of claim 3, wherein when the sample gene expression profile is classified as:
(i) insulinoma-like, the step (d) of providing a prediction of prognosis comprises prediction of a good prognosis;
(ii) intermediate, the step (d) of providing a prediction of prognosis comprises prediction of a good prognosis;
(iii) MLP, the step (d) of providing a prediction of prognosis comprises prediction of a poor prognosis.

9. The method of claim 1, wherein step b) making a prediction of the prognosis of the patient based on the sample gene expression profile comprises:
(i) optionally, normalising the measured expression level of each gene relative to the expression level of one or more housekeeping genes;
(ii) comparing the sample gene expression profile, optionally after said normalising, with the expression profile of:
a high risk control group of PanNET patients known to have had a median overall survival time post-diagnosis of less than 71 months, or even less than 60 months; and
a low risk control group of PanNET patients known to have had a median overall survival time post-diagnosis of greater than 71 months, or even more than 100 months;
c) classifying the sample gene expression profile as belonging to the risk group having the gene expression profile to which it is most closely matched; and
d) providing a prediction of prognosis based on the classification made in step c).

10. The method of claim 9, wherein step (ii) of comparing the sample gene expression profile comprises comparing the sample gene expression profile, with at least two reference centroids corresponding to low and high risk subgroups, respectively, the reference centroid comprising:
a first reference centroid that represents the summarised gene expression of the high risk patients measured in a high risk training set made up of PanNET patients known to have had a median overall survival time post-diagnosis of less than 71 months, or even less than 60 months;
a second reference centroid that represents the summarised gene expression of the low risk patients measured in a low risk training set made up of PanNET patients known to have had a median overall survival time post-diagnosis of greater than 71 months, or even more than 100 months.

11. The method of claim 3, wherein the sample gene expression profile is compared with each reference centroid for closeness of fit using Persons correlation.

12. The method of claim 1, comprising the additional step of identifying any mutations within one of more of the genes selected from: MEN1, ATRX, DAXX, PTEN, TSC1, TSC2 and ATM in a sample obtained from the PanNET of the patient,
wherein step (b) involves making a making a prediction of the prognosis of the patient based on the sample gene expression profile and optionally the mutation status of the one or more genes.

13. The method of claim 12, wherein the presence of a mutation in MEN1 is indicative of the PanNET being intermediate subtype.

14. The method of claim 12, wherein when a mutation in MEN1 is identified in the PanNET:
(i) the patient is at low risk of poor prognosis; and/or
(ii) the patient is predicted to have a good prognosis.

15. The method of claim 12 wherein the presence of a mutation in DAXX and/or ATRX is indicative of the PanNET being intermediate subtype or MLP subtype.

16. The method of claim 12 wherein the presence of a mutation in TSC2, PTEN and/or ATM is indicative of the PanNET being intermediate subtype or MLP subtype.

17. The method of claim 1, wherein the patient, having been determined to be at high risk of poor prognosis, is selected for additional or alternative treatment, including aggressive treatment, optionally, wherein the patient is selected for treatment with one or more of: platinum-based chemotherapy doublets, sunitinib, everolimus, peptide receptor radionuclide therapy (PRRT), chemotherapy, and therapeutic trials.

18. The method of claim 1, wherein the patient, having been found to be at low risk of poor prognosis, is selected less aggressive ongoing treatment or for monitoring or non-treatment, optionally wherein the patient is selected for non-treatment and monitoring, or treatment by somatostatin analogues.

19. The method of claim 1, wherein the PanNET in the patient has already been classified as grade 1/2 according to the WHO classification system.

20. The method according to claim 19, wherein if the sample gene expression profile is classified as MLP, or as high risk, the patient is at high risk of poor prognosis.

21. The method of claim 1, wherein the PanNET in the patient has already been classified as grade 3 according to the WHO classification system.

22. The method according to claim 21, wherein if the sample gene expression profile is classified as intermediate, insulinoma-like, or as low risk, the patient is at low risk of poor prognosis.

23. A computer-implemented method for predicting the prognosis of a human PanNET patient, the method comprising:
a) obtaining gene expression data comprising a gene expression profile representing gene expression measurements of at least 30 genes selected from: CEACAM1, INS, PFKFB2, ELSPBP1, MIA2, ENTPD3, GRM5, STEAP3, APOH, SERPINA1, A1CF, PRLR, F10, TMEM176B, MASP2, RBP4, CYP4F3, CHST8, KLK4, USP29, CELA1, TM4SF4, TMPRSS4, SCD5, TM4SF5, SERPIND1, P2RX1, GLP1R, LRAT, CASR, DAPL1, ERBB3, C19orf77, F7, PLIN3, NEFM, MNX1, ROBO3, CPA1, CTRL, TGFBR3, PNLIPRP2, TSHZ3, ADAMTS2, GLRA2, HGD, GP2, CTRC, RAB17, ANGPTL3, LOXL4, PNLIP, PEMT, CPA2, PNLIPRP1, ALDH1A1, SLC12A7, IL20RA, CLPS, GLS, C20orf46, GCGR, IL18R1, PDIA2, NAAA, BTC, TAPBPL, ELMO1, KLK8, CDS1, TFF1, TBC1D24, KIT, MOBKL1A, PLA1A, SUSD5, CRYBA2, PMM1, EFNA1, SLC16A3, FKBP11, IL22RA1, ADM, EGLN3, LGALS4, TLE2, CLDN10, NUPR1, SERPINI2, PTPLA, PVRL4, EGFR, MAFB, PFKFB3, HSD11B2, FGB, NDC80, SMOC2, ACVR1B, TGIF1, ARRDC4, MMP1, TACSTD2, TOP2A, SH3BP4, PDGFC, THBS2, CNPY2, HAO1, ADAM28, C7orf68, GATM, CXCR4, PAFAH1B3, NEK6, AKR1C4, F12, PMEPA1, RAB7L1, SMO, CLDN1, CHST1, WNT4, TMPRSS15, SPAG4, MX2, SLC7A2, GUCA1C, SLC7A8, PRSS22, RARRES2, PRSS8, SLC30A2, TMEM90B, VIPR2, CXCR7, SMARCA1, FAM19A5, CLDN11, SERPINA3, GAL3ST4, AFG3L1, COL8A1, SSX2IP, IMPA2, VEGFC, TMEM181, LGALS2, PLXDC1, TLR3, PSMB9, CHI3L2, PLCE1, ABI3BP, NUDT5, FOXO4, SLC2A1, COL1A2, REG1B, NETO2, ENC1, DLL1, TM4SF1, CKS2, FGD1, PPEF1, LEF1, MLN, TNFAIP6, ACAD9, TYMS, ZNF521, ACADSB, TSC2, HR, DEFB1, GRSF1, ACE, SRGAP3, SMEK1, TWIST1, FMNL1, ADAMTS7, COL5A2, IFI44, CAPN13, AQP8, IP6K2, COPE, MXRA5, RBPJL, MBP, MAP3K14, CLCA1, IDS, TECR, CAPNS1, POSTN, measured in a sample obtained from the PanNET of the patient; and
b) (i) optionally, normalising the measured expression level of each gene relative to the expression level of one or more housekeeping genes,
(ii) comparing the sample gene expression profile with two or more reference centroids as defined in claim 3;
c) classifying the sample gene expression profile as belonging to the risk group having the reference centroid to which it is most closely matched; and
d) providing a prediction of prognosis based on the classification made in step c).

24. A method of treatment of PanNET in a human patient, the method comprising:
(a) carrying out the method of claim 1; and
(b) (i) when the patient is determined to be at high risk of poor prognosis, or is predicted to have a poor prognosis, administering additional anti-tumor therapy or more aggressive anti-tumor therapy; or
(ii) when the patient is determined to be at low risk of poor prognosis, or is predicted to have a good prognosis, not administering additional anti-tumor therapy or administering anti-tumor therapy that is less aggressive.

25. A method according to claim 24, wherein when the patient is determined to be at high risk of poor prognosis, or is predicted to have a poor prognosis, the patient is selected for treatment with one or more of: platinum-based chemotherapy doublets, sunitinib, everolimus, peptide receptor radionuclide therapy (PRRT), chemotherapy, and therapeutic trials.

26. A method according to claim 24, wherein when the patient is determined to be at low risk of poor prognosis, or is predicted to have a good prognosis, the patient is selected for non-treatment and monitoring, or treatment by somatostatin analogues.
